(19) United States
(12) Patent Application Publication
(10) Pub. No.: US 2024/0252026 A1
(43) Pub. Date: Aug. 1, 2024

(54) ARTICULATION DEVICES, SYSTEMS, AND METHODS

(71) Applicant: Biomerics, LLC, Salt Lake City, UT (US)

(72) Inventors: David P. Hornak, Monroe, CT (US); John J. Franchi, Guilford, CT (US); Xiao Wu, Orange, CT (US); Robert F. Smith, Waterbury, CT (US)

(73) Assignee: Biomerics, LLC, Salt Lake City, UT (US)

(21) Appl. No.: 18/634,936

(22) Filed: Apr. 13, 2024

Related U.S. Application Data
(63) Continuation of application No. PCT/US22/46628, filed on Oct. 13, 2022.
(60) Provisional application No. 63/255,018, filed on Oct. 13, 2021.

(51) Int. Cl. A61B 1/005; A61B 1/00

(52) U.S. Cl. CPC A61B 1/0055; A61B 1/00128; A61B 1/0057

(57) ABSTRACT

A backbone for use in an endoscope can include a plurality of links of which adjacent pairs are interconnected with each other. Each of the plurality of links can include an inner link segment and an outer link segment. One of the inner link segment and the outer link segment can include a pivot member and a socket. The other of the inner link segment and the outer link segment can include a locking member and a recess. The pivot member of a first link of the plurality of links can be received into the socket of a second link of the plurality of links. The locking member of the first link can be received into the recess of the second link.

### CROSS-REFERENCE TO RELATED APPLICATIONS

This application is a continuation of World Intellectual Property Organization International Application No. PCT/US2022/046628, filed on Oct. 13, 2022, titled ARTICULATION DEVICES, SYSTEMS, AND METHODS, which claims the benefit of U.S. Provisional Patent Application No. 63/255,018, filed on Oct. 13, 2021, titled ARTICULATION DEVICES, SYSTEMS, AND METHODS, the entire contents of each of which are hereby incorporated by reference herein.

### TECHNICAL FIELD

Certain embodiments described herein relate generally to endoscopes and other elongated instruments and related methods for manufacturing the same, and further embodiments relate more particularly to bending sections and bending section components for, e.g., endoscopes and related methods for manufacturing and using the same.

### BACKGROUND

Known endoscopes and other elongated medical devices suffer from a variety of drawbacks and/or would benefit from improvements. Embodiments disclosed herein remedy, ameliorate, or avoid one or more of such drawbacks and/or include improvements relative to known endoscopes and/or other elongated medical devices, such as steerable medical devices, and/or bending sections therefor.

### BRIEF DESCRIPTION OF THE DRAWINGS

The written disclosure herein describes illustrative embodiments that are non-limiting and non-exhaustive. Reference is made to certain of such illustrative embodiments that are depicted in the figures, in which:

FIG. 1 is an elevation view of an embodiment of an endoscope that includes a bending section capable of bending in four separate directions;

FIG. 2A is a perspective view of an embodiment of an articulable backbone compatible with the bending section of an elongated medical device, such as, e.g., the endoscope of FIG. 1, that includes a plurality of interconnected links;

FIG. 2B is a front elevation view of the backbone of FIG. 2A;

FIG. 2C is a side elevation view of the backbone of FIG. 2A, the view being rotated 90 degrees clockwise (in a proximal-to-distal reference frame) about a longitudinal axis of the backbone relative to the view depicted in FIG. 2B;

FIG. 3 is a partially exploded front elevation view of the backbone of FIG. 2A with a set of outer link segments shown separated from a set of inner link segments;

FIG. 4A is a perspective view of an embodiment of an inner link segment that is compatible with the backbone of FIG. 2A;

FIG. 4B is a front elevation view of the inner link segment of FIG. 4A;

FIG. 4C is a side elevation view of the inner link segment of FIG. 4A that is rotationally offset about a longitudinal axis of the inner link by 90 degrees relative to the view depicted in FIG. 4B;

FIG. 5A is a perspective view of another embodiment of an inner link segment that is compatible with the backbone of FIG. 2A and that includes a plurality of guides for the passage of guidewires;

FIG. 5B is a front elevation view of the inner link segment of FIG. 5A;

FIG. 5C is a side elevation view of the inner link segment of FIG. 5A that is rotationally offset about a longitudinal axis of the inner link segment by 90 degrees relative to the view depicted in FIG. 5B;

FIG. 6A is a perspective view of an embodiment of an inner pull ring segment that is compatible with the backbone of FIG. 2A;

FIG. 6B is a front elevation view of the inner pull ring segment of FIG. 6A;

FIG. 6C is a side elevation view of the inner pull ring segment of FIG. 6A that is rotationally offset about a longitudinal axis of the inner pull ring segment by 90 degrees relative to the view depicted in FIG. 6B;

FIG. 7A is a perspective view of an embodiment of an outer link segment that is compatible with the backbone of FIG. 2A;

FIG. 7B is a front elevation view of the outer link segment of FIG. 7A;

FIG. 7C is a side elevation view of the outer link segment of FIG. 7A that is rotationally offset about a longitudinal axis of the outer link segment by 90 degrees relative to the view depicted in FIG. 7B;

FIG. 8A is a perspective view of an embodiment of an outer pull ring segment that is compatible with the backbone of FIG. 2A;

FIG. 8B is a front elevation view of the outer pull ring segment of FIG. 8A;

FIG. 8C is a side elevation view of the outer pull ring segment of FIG. 8A that is rotationally offset about a longitudinal axis of the outer pull ring segment by 90 degrees relative to the view depicted in FIG. 8B;

FIG. 9 is a perspective view of the backbone of FIG. 2A with only a pull ring and three distalmost links thereof shown coupled with a plurality of pull wires;

FIG. 10A is a front elevation view of a pair of interconnected links shown in a straight or unarticulated state;

FIG. 10B is a front elevation view of a pair of inner link segments associated with the interconnected links of FIG. 10A shown in the straight or unarticulated state;

FIG. 10C is a front elevation view of a pair of outer link segments associated with the interconnected links of FIG. 10A shown in the straight or unarticulated state;

FIG. 11A is a front elevation view of the pair of interconnected links of FIG. 10A shown in an illustrative articulated state;

FIG. 11B is a front elevation view of the pair of inner link segments associated with the interconnected links of FIG. 10A shown in the articulated state of FIG. 11A;

FIG. 11C is a front elevation view of the pair of outer link segments associated with the interconnected links of FIG. 10A shown in the articulated state of FIG. 11A;

FIG. 12A is a front elevation view of the pair of interconnected links of FIG. 10A shown in another illustrative articulated state in which an articulation limit has been reached;

FIG. 12B is a front elevation view of the pair of inner link segments associated with the interconnected links of FIG. 10A shown in the articulated state of FIG. 12A;

FIG. 12C is a front elevation view of the pair of outer link segments associated with the interconnected links of FIG. 10A shown in the articulated state of FIG. 12A;

FIG. 13 is a side elevation view of a second pair of interconnected links shown in an articulated state such as that depicted in FIGS. 12A-12C, in which the articulation limit has been reached, the view being rotated 90 degrees clockwise (in a proximal-to-distal reference frame) relative to the view depicted in FIG. 12A;

FIG. 14A is a front perspective view of six interconnected links shown in an articulated state, wherein three distinct link doublets are shown, with each doublet having rotated in unison in a first dimension to define a first arc;

FIG. 14B is a side perspective view of the six interconnected links of FIG. 14A shown in the articulated state, the view being rotated approximately 90 degrees counterclockwise (in a proximal-to-distal reference frame) about a longitudinal axis of the backbone relative to the view depicted in FIG. 14A, wherein two distinct link doublets are shown, with each doublet having rotated in unison in a second dimension to define a second arc;

FIG. 15A is a front elevation view of another embodiment of an articulable backbone that includes a plurality of interconnected links;

FIG. 15B is a side elevation view of the backbone of FIG. 15A that is rotationally offset about a longitudinal axis of the backbone by 90 degrees relative to the view depicted in FIG. 15A;

FIG. 16 is a perspective view of another embodiment of an articulable backbone that includes a plurality of interconnected links;

FIG. 17A is a perspective view of an embodiment of an inner guide link segment that is compatible with the backbone of FIG. 16;

FIG. 17B is a front elevation view of the inner guide link segment of FIG. 17A;

FIG. 17C is a side elevation view of the inner guide link segment of FIG. 17A that is rotationally offset about a longitudinal axis of the inner guide link by 90 degrees relative to the view depicted in FIG. 17B;

FIG. 18 is a top plan view of an embodiment of a guide link within the backbone of FIG. 16, the guide link including the inner guide link segment of FIG. 17A and including pull wires extending therethrough, wherein a cylindrical accessory space is depicted in broken lines;

FIG. 19 is a perspective view of another embodiment of an articulable backbone that includes a plurality of interconnected links;

FIG. 20A is a perspective view of an embodiment of an inner guide link segment that is compatible with the backbone of FIG. 19;

FIG. 20B is a front elevation view of the inner guide link segment of FIG. 20A;

FIG. 20C is a side elevation view of the inner guide link segment of FIG. 20A that is rotationally offset about a longitudinal axis of the inner guide link by 90 degrees relative to the view depicted in FIG. 20B;

FIG. 20D is another side elevation view of the inner guide link segment of FIG. 20A that is rotationally offset about a longitudinal axis of the inner guide link by 180 degrees relative to the view depicted in FIG. 20C;

FIG. 21A is a top plan view of an embodiment of a guide link within the backbone of FIG. 19, the guide link including the inner guide link segment of FIG. 20A and including pull wires extending therethrough, wherein a substantially cylindrical accessory space is depicted in broken lines;

FIG. 21B is a top plan view of another guide link that is of the same variety as and is longitudinally spaced from the guide link of FIG. 21A within the backbone of FIG. 19, the guide link including pull wires extending therethrough, wherein the substantially cylindrical accessory space is depicted in broken lines;

FIG. 22A is a perspective view of a portion of a hypotube illustrating a stage of an illustrative method of forming a backbone;

FIG. 22B is a perspective view of a portion of a further hypotube illustrating another stage of the illustrative method of forming the backbone;

FIG. 22C is a perspective view of the portion of the hypotube of FIG. 22A having been laser cut in another stage of the illustrative method of forming the backbone;

FIG. 22D is a perspective view of the portion of the further hypotube of FIG. 22B illustrating another stage of the illustrative method of forming the backbone;

FIG. 23A depicts an inner pull ring member in a stage of an illustrative method of assembling an embodiment of a backbone;

FIG. 23B depicts an outer pull ring member being advanced over the inner pull ring member in another stage of the illustrative method of assembling an embodiment of a backbone;

FIG. 23C depicts an alignment rod being advanced through the inner and outer pull ring members in another stage of the illustrative method of assembling an embodiment of a backbone;

FIG. 23D depicts an inner link member being advanced distally and then laterally through sockets in the inner and outer pull ring members to be coupled with the socket of the inner pull ring member in another stage of the illustrative method of assembling an embodiment of a backbone;

FIG. 23E depicts an outer link member being advanced distally over the inner link member in another stage of the illustrative method of assembling an embodiment of a backbone;

FIG. 23F depicts an alignment rod being advanced through the inner and outer link members in another stage of the illustrative method of assembling an embodiment of a backbone;

FIG. 23G depicts a series of additional links having been coupled in like manner to the pull ring members, each pair of link members and pull ring members having been welded, and pull wires having been coupled with the backbone in another stage of the illustrative method of assembling an embodiment of a backbone;

FIG. 24 is a perspective view of another embodiment of an articulable backbone that includes a plurality of interconnected links;

FIG. 25A is a front elevation view of an embodiment of an inner guide link segment that is compatible with the backbone of FIG. 24;

FIG. 25B is a front elevation view of another embodiment of an inner guide link segment that is compatible with the backbone of FIG. 24, wherein the inner guide link segments of FIGS. 25A and 25B are mirror images of one another across a central longitudinal plane;

FIG. 26A is a top plan view of an embodiment of a guide link within the backbone of FIG. 24, the guide link including an inner guide link segment such as that of FIG. 25A and including pull wires extending therethrough, wherein a substantially cylindrical accessory space is depicted in a first set of broken lines and a substantially elliptic cylindrical space is depicted in a second set of broken lines;

FIG. 26B is a top plan view of another embodiment of a guide link within the backbone of FIG. 24 that is longitudinally spaced from the guide link of FIG. 26A, the guide link including an inner guide link segment such as that of FIG. 25B and including pull wires extending therethrough, wherein the substantially cylindrical accessory space is depicted in broken lines and the substantially elliptic cylindrical space is depicted in the second set of broken lines;

FIG. 26C is a top plan view of another embodiment of a guide link within the backbone of FIG. 24 that is longitudinally spaced from the guide link of FIG. 26B, the guide link including an inner guide link segment such as that of FIG. 25A, but rotated 180 degrees relative to the orientation of FIG. 26A, wherein the substantially cylindrical accessory space is depicted in broken lines and the substantially elliptic cylindrical space is depicted in the second set of broken lines;

FIG. 26D is a top plan view of another embodiment of a guide link within the backbone of FIG. 24 that is longitudinally spaced from the guide link of FIG. 26C, the guide link including an inner guide link segment such as that of FIG. 25B, but rotated 180 degrees relative to the orientation of FIG. 26B, wherein the substantially cylindrical accessory space is depicted in broken lines and the substantially elliptic cylindrical space is depicted in the second set of broken lines;

FIG. 27A is a perspective view of another embodiment of an inner guide link that is compatible with various embodiments of articulable backbones;

FIG. 27B is a bottom plan view of the inner guide link of FIG. 27A;

FIG. 28A is a perspective view of another embodiment of an inner guide link that is compatible with various embodiments of articulable backbones;

FIG. 28B is a bottom plan view of the inner guide link of FIG. 28A;

FIG. 29A is a perspective view of another embodiment of an inner guide link that is compatible with various embodiments of articulable backbones;

FIG. 29B is a bottom plan view of the inner guide link of FIG. 29A;

FIG. 30 is a perspective view of another embodiment of an articulable backbone compatible with the bending section of an elongated medical device, such as, e.g., the endoscope of FIG. 1, that includes a plurality of interconnected links;

FIG. 31 is a perspective view of an embodiment of an outer guide link that is compatible with, e.g., the backbone of FIG. 30;

FIG. 32A is a front elevation view of the outer guide link of FIG. 31;

FIG. 32B is another elevation view of the outer guide link of FIG. 31 at a 90-degree rotation relative to FIG. 32A;

FIG. 32C is another elevation view of the outer guide link of FIG. 31 at a 180-degree rotation relative to FIG. 32A;

FIG. 32D is another elevation view of the outer guide link of FIG. 31 at a 270-degree rotation relative to FIG. 32A;

FIG. 33A is a perspective view of an embodiment of an inner guide link that is compatible with, e.g., the backbone of FIG. 30;

FIG. 33B is a front elevation view of the inner guide link of FIG. 33A;

FIG. 33C is another elevation view of the inner guide link of FIG. 33B at a 90-degree rotation relative to FIG. 33B;

FIG. 34A is a perspective view of an assembled guide link that includes both the outer guide link of FIG. 31 and the inner guide link of FIG. 33A;

FIG. 34B is a front elevation view of the assembled guide link of FIG. 34A;

FIG. 35 is a front elevation view of a pair of assembled guide links demonstrating interconnection of adjacent guide links;

FIG. 36 is a perspective view of an embodiment of an inner proximal link that is compatible with, e.g., the backbone of FIG. 30;

FIG. 37A is a perspective view of an embodiment of an outer proximal link that is compatible with, e.g., the backbone of FIG. 30;

FIG. 37B is another perspective view of the outer proximal link of FIG. 37A;

FIG. 38 is a perspective view of an embodiment of an inner pull ring or inner distal link that is compatible with, e.g., the backbone of FIG. 30;

FIG. 39 is a perspective view of an embodiment of an outer pull ring or outer distal link that is compatible with, e.g., the backbone of FIG. 30;

FIG. 40 is a perspective view of another embodiment of an articulable backbone compatible with the bending section of an elongated medical device, such as, e.g., the endoscope of FIG. 1, that includes a plurality of interconnected links; and

FIG. 41 is a perspective view of a front half (with the back half omitted for visual clarity) of an embodiment of an outer guide link that is compatible with, e.g., the backbone of FIG. 40.

### DETAILED DESCRIPTION

The present disclosure relates generally to endoscopes and other elongated medical instruments having deflectable ends. Some embodiments, in particular, relate to articulation devices, systems, and methods for endoscopes. Various embodiments include, for example, endoscopes that include articulable backbone structures, backbone structures and/or components thereof suitable for use in deflectable distal ends of endoscopes, and methods of manufacturing and using each of the foregoing.

Various known endoscopes include bending sections that permit selective steering and/or deflection of a distal end of the endoscope. In certain bending sections, a backbone may include a plurality of interconnected segments that permit relative rotation between adjacent components. In certain devices, particularly in devices that permit four-way bending of the bending section (i.e., in which the distal end may be curved in either of two oppositely directed primary directions and either of two oppositely directed lateral directions, with the primary and lateral directions being offset by 90 degrees, or stated otherwise, being orthogonally oriented), the backbone can be complex and/or can be costly to manufacture and/or assemble. In some instances, the backbone can be constructed from components that are machined, stamped, formed by metal injection molding (MIM), etc., and in further instances, may be riveted or pinned together or otherwise connected into complex assemblies. In other or further instances, a separate pull ring member to which pull wires are attached may be complicated and/or may require the use of specialized and/or costly procedures in coupling the wires thereto and/or the coupling thereof with the rest of the backbone. The manufacturing costs of such assemblies can be substantial. For example, the costs can be highly prohibitive for single-use or disposable endoscopes.

Various embodiments disclosed herein can ameliorate or remedy one or more drawbacks or limitations of known endoscopes and known articulation components thereof. For example, various embodiments disclosed herein can be manufactured relatively easily, economically, and/or at a price suitable for disposability. Other or further embodiments can facilitate design processes. Still other or further embodiments can provide for efficient and/or reliable assembly. Still other or further embodiments can provide robust bending sections that can readily withstand stresses that can arise during use of an endoscope or other elongated instrument, such as may result from tensioning from one or more pull wires and/or forces imparted by anatomical features. One or more of these and/or other advantageous features of embodiments described herein will be apparent from the present disclosure.

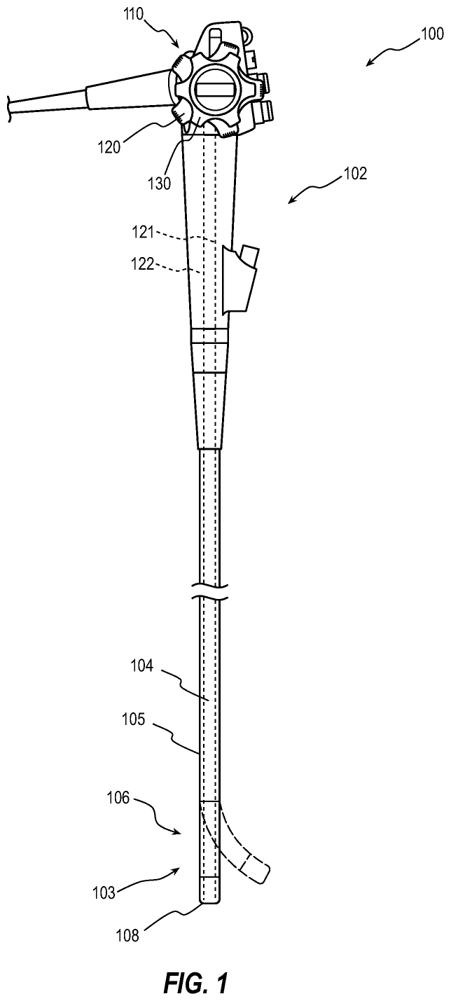

With reference to FIG. 1, in certain embodiments, an endoscope 100 includes a handle 102 and an elongated shaft 104. The shaft 104 can be configured to be introduced into a patient via any suitable lumen, whether naturally present or surgically formed. The shaft 104 can include a bending section 106 that is positioned at or near a distal end 103 of the shaft 104. The shaft 104 can further include a tip member or tip 108, which may be positioned at a distalmost extremity of the shaft 104. In various embodiments, the tip 108 and/or other portions of the distal end 103 of the shaft 104 can be configured for any of a variety of functions, including those known in the art and those yet to be devised. For example, in various embodiments, the tip 108 may include one or more of image gathering apparatus (e.g., a camera, such as a CCD device), one or more lighting features (e.g., LEDs, optical fibers, and/or optical lenses and/or other componentry), an irrigation port, and/or a working channel port through which instruments may be extended from the distal end of the endoscope 100, such as, e.g., past a distal tip of the endoscope 100. In certain embodiments in which a working channel is present, the working channel may extend through an entirety of or through substantially all of the shaft 104, and may, in further embodiments, additionally extend through at least a portion of the handle 102. For example, a proximal end of the working channel can be accessible at or via the handle 102.

In various embodiments, the handle 102 can include a deflection or steering mechanism 110 via which deflection or steering of the bending section 106 can be achieved. In particular, the steering mechanism 110 can be manipulated or actuated to deflect, redirect, point, steer, or otherwise selectively position or reposition the distal tip 108 of the shaft 104. The steering mechanism 110 can include one or more actuators 120, 130 that can be manipulated or actuated to control such movement of the distal tip 108. For example, in some embodiments, the actuator 120 can be mechanically connected to one or more pull wires of any suitable variety and in any suitable manner.

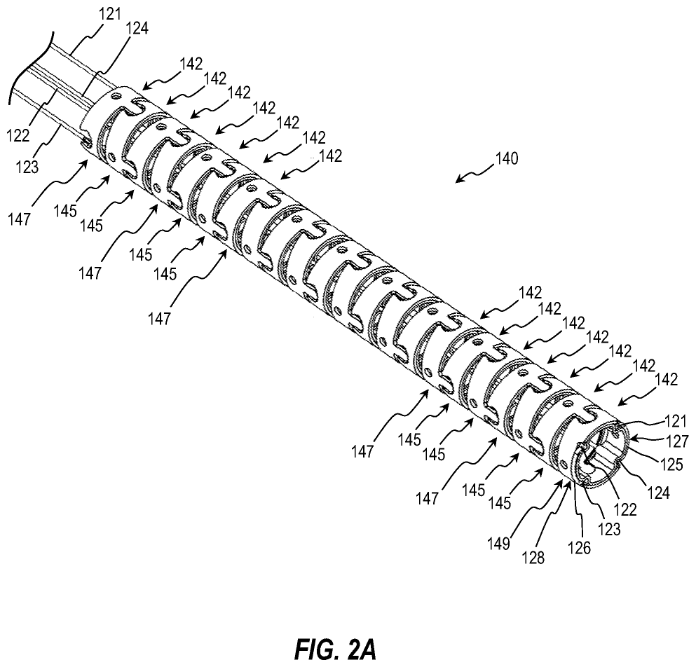

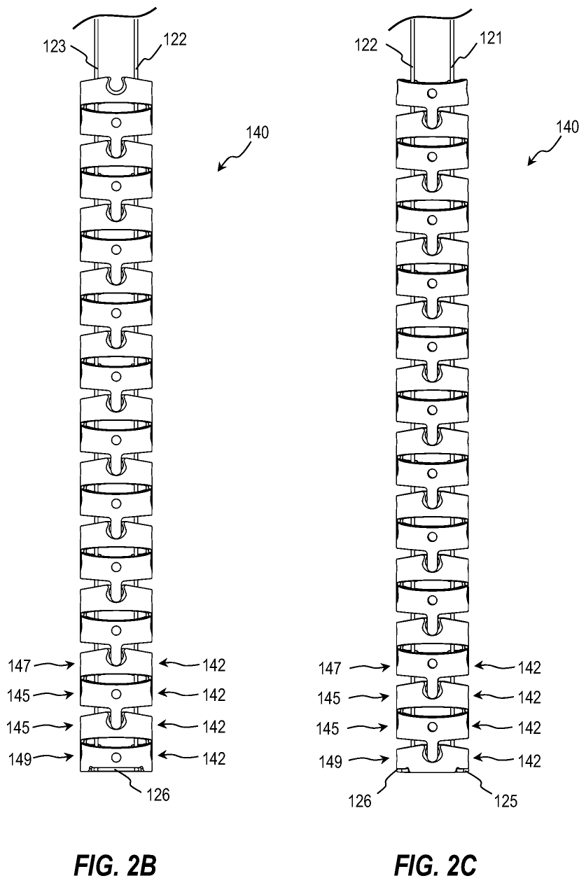

With reference to FIGS. 1-2C, the illustrated embodiment includes four pull wires 121, 122, 123, 124 (the pull wires 123, 124 are obscured in FIG. 1, but are visible in, e.g., FIG. 2A). Each of the pull wires 121, 122, 123, 124 is angularly spaced from each adjacent wire by 90 degrees. Stated otherwise, the pull wires 121, 122, 123, 124 are angularly offset from one another by 90 degrees in the illustrated embodiment. In some embodiments, two or more of the pull wires 121, 122 may be separate sections of a single unitary wire. For example, as shown in FIG. 2A, in the illustrated embodiment, the pull wires 121, 124 are separate lengths of a unitary wire 127. A connector wire 125 can extend between and join the distal ends of the pull wires 121, 124. Similarly, the pull wires 122, 123 are separate lengths of a unitary wire 128. A connector wire 126 can extend between and join the distal ends of the pull wires 122, 123. The pull wires 121, 122, 123, 124 can be connected to the distal end 103 (FIG. 1) of the shaft 104. For example, in some embodiments, the distal ends of the pull wires 121, 122, 123, 124 can be connected to the shaft 104 at a distal end of the bending section 106, and may, in other or further instances, be positioned at or near the distal tip 108.

With continued reference to FIGS. 1-2C, rotation of the actuator 120 in a first direction (e.g., counterclockwise, in the view illustrated in FIG. 1) can pull on or apply tension to one or more of the pull wires 121, 122, 123, 124 and release tension on or slacken one or more of the remaining pull wires. This can deflect the distal end of the shaft 104 in a first direction, such as to the right in the illustrated view, as shown in broken lines. Rotation of the actuator 120 in a second direction opposite the first direction (e.g., clockwise, in the view illustrated in FIG. 1), can reverse the tensioning and slackening of the pull wires and effect deflection of the distal end of the shaft 104 in the opposite direction, or to the left in the illustrated view. The first and second deflection directions of the distal end of the shaft 104 may be within the same plane. For example, in the illustrated view of FIG. 1, oppositely directed rotations of the actuator 120 can sweep the distal end of the shaft 104 through the first and second directions (right and left) within the plane of the page. In some instances, this form of deflection may be referred to as “up/down” deflection.

In some embodiments, oppositely directed rotations of the actuator 130 can achieve lateral deflections of the distal end of the shaft 104 in third and fourth directions (e.g., which may be opposite from each other) that are angularly offset from the first and second deflection directions that are achieved via the actuator 120. For example, in some embodiments, the third and fourth deflection directions achievable via the actuator 130 may deflect the distal end of the shaft 104 within a plane that is orthogonal to the plane of the page and that is aligned with the longitudinal axis of the shaft 104 (e.g., in directions into and out of the plane of the page). In some instances, this form of deflection may be referred to as “left/right” deflection. Stated otherwise, in some embodiments, the steering mechanism 110, which can include the actuators 120, 130 coupled with a plurality of pull wires, can achieve deflection of the distal end of the shaft 104 in four mutually orthogonal directions.

With reference to FIGS. 2A-2C, in certain embodiments, a backbone 140, which may additionally or alternatively be referred to as a spine, articulation column, etc., can be included within the bending region 106 of the shaft 104. For example, in some embodiments, the shaft 104 may include a substantially continuous outer layer or sleeve 105 (FIG. 1) that spans a full or substantially full length of the shaft 104. The sleeve 105 may extend over an exterior of the backbone 140 at the bending region 106.

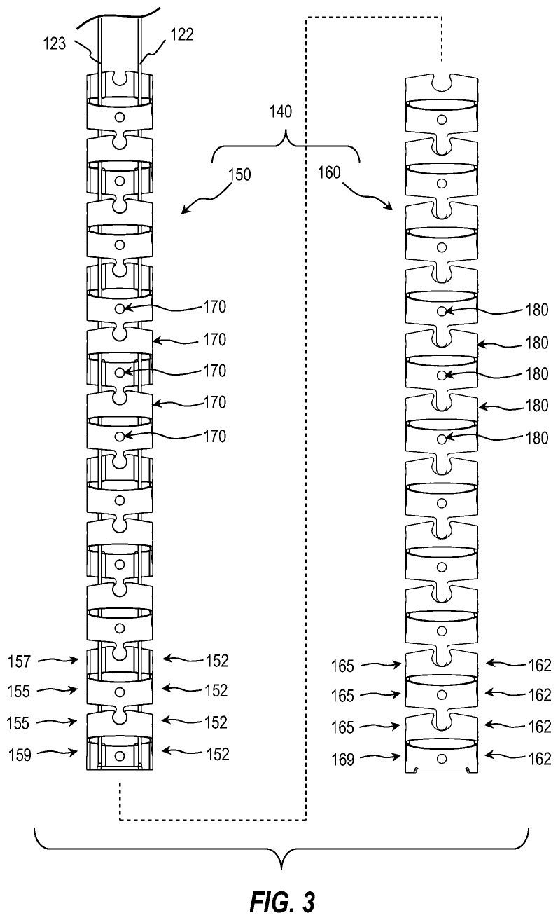

The backbone 140 can include a plurality of interconnected links 142, which may additionally or alternatively be referred to as vertebrae, sections, rings, etc. In some embodiments, different types of links 142 may be present in the backbone 140. For example, the illustrated embodiment includes three different types of links: fourteen basic links 145, seven guide links 147, and one terminal link 149. The positions of the differing links are readily perceptible in, e.g., FIGS. 2A and 3. As further discussed below, the basic links 145 may have little or no direct interaction with the guidewires beyond, in some instances, contact therewith at an internal surface of the basic links 145; the guide links 147 may include guides through which the guidewires may pass, be received, or extend; and the terminal link 149 may be fixedly secured to the guidewires. For example, as shown in FIG. 2A, the guidewires 121, 122, 123, 124 are fixedly secured to the terminal link 149 at their respective distal ends. The terminal link 149 can be positioned at the distal end of the backbone 140, and may alternatively be referred to as the pull ring 149. Any suitable number of links 142 is contemplated. Moreover, any suitable combination of the various types of links 145, 147, 149 is contemplated. For example, whereas basic links 145 and guide links 147 are present in the illustrated embodiment in a ratio of 2:1, any other ratio is contemplated, including, e.g., 1:1, 3:1, 4:1, 5:1, 6:1. Other relative numbers of the links 145, 147 and/or patterns of their alternation are also contemplated. In still other embodiments, no basic links 145 may be present. For example, in some embodiments, a backbone 140 includes only guide links 147. In other embodiments, a backbone 140 includes only two types of links—e.g., guide links 147 and a terminal link or pull ring 149 at a distal end of the backbone 140.

With reference to FIG. 3, the backbone 140 can be formed of an inner backbone 150 and an outer backbone 160 joined together. Stated otherwise, each link 142 of the backbone 140 can be formed of an inner link 152 and an outer link 162 that are joined together. The inner and outer links 152, 162 may alternatively be referred to as inner and outer link segments, but for convenience of disclosure herein, will be referred to by the shorter appellation of “links.” Accordingly, other instances of the term “link,” as applied to a member of a composite link 142, may be interchanged with the term “link segment.” As further discussed below, the pairs of inner and outer links 152, 162 can be fixedly secured together in any suitable manner, such as via welding. In the illustrated embodiment, micro welding is used to secure the inner and outer links 152, 162 together.

Each of the inner links 152 of the illustrated embodiment is one of three varieties: an inner basic link 155, an inner guide link 157, and an inner terminal link or inner pull ring 159. Each of the outer links 162 of the illustrated embodiment, however, is one of only two varieties: an outer basic link 165 and an outer terminal link or outer pull ring 169. Stated otherwise, in the illustrated embodiment, an outer basic link 165 can be compatible with each of two varieties of inner links: namely, the inner basic link 155 variety and the inner guide link 157 variety. In the illustrated embodiment, each link component may be identical or substantially identical to all other link components of the same variety. Thus, for example, each outer basic link 165 may be substantially identical to all other outer basic links 165 of the outer backbone 160.

With continued reference to FIG. 3, and in view of FIGS. 2A-2C, each basic link 145 of the backbone 140 can be formed from an inner basic link 155 joined to an outer basic link 165, each guide link 147 of the backbone 140 can be formed from an inner guide link 157 joined to an outer basic link 165, and the pull ring 149 of the backbone 140 can be formed from an inner pull ring 159 joined to an outer pull ring 169.

In some embodiments, all of the inner links 152 can be formed from a single tube, such as, for example, a thin-walled metallic hypotube. The inner tube can be laser cut to form the inner links 152. In certain of such embodiments, indentations can be formed in a sidewall of the inner guide links 157 and in a sidewall of the inner pull ring to form channels for passage of the guidewires 121, 122, 123, 124 (as discussed further below). In some instances, the indentations are formed prior to laser cutting the tube. In other instances, the indentations are formed after the tube has been laser cut to form the inner links 152. The channels may also be referred to as guides.

In other embodiments, the inner links of different varieties may be formed from one or more separate tubes. For example, the inner basic links 155 may be formed from a first tube and the inner guide links 157 may be formed from a second tube. In various embodiments, the inner pull ring 159 may also be formed from the second tube, or it may be formed from a third tube. The second and third tubes may be indented with four parallel elongated indentations prior to laser cutting to form the channels for the passage of pull wires, or the individual links formed via the laser cutting may be indented after the laser cutting.

Similarly, in some embodiments, all of the outer links 160 can be formed from a single tube, such as, for example, a thin-walled metallic hypotube. The outer tube can be laser cut to form the outer links 162. In other embodiments, the outer basic links 165 may be formed from a first tube and the outer pull ring 169 can be formed from a separate second tube.

With continued reference to FIG. 3, in some embodiments, each of the inner and links 152 can include alignment openings 170 and each of the outer links 162 can include alignment openings 180. After the inner and outer links 152, 162 have been cut, the inner links 152 can be inserted into the outer links 162, whether individually or in a group, and the alignment openings 170, 180 of the inner and outer links 152, 162, respectively, can be aligned with each other. For example, in some embodiments, the inner and outer alignment openings 170, 180 are substantially the same size, such that when the inner and outer openings 170, 180 are aligned, the perimeters of the inner and outer openings 170, 180 define a substantially continuous cylindrical channel. In some instances, welding (for example, micro welding, micro resistance welding, laser spot welding, etc.) may take place at one or both sets of aligned openings 170, 180. In other instances, an alignment rod (not shown) may be inserted through both sets of aligned openings 170, 180 to maintain proper orientation of the inner and outer links 152, 162. Welding of the inner and outer links 152, 162 may take place at some other position—that is, at any suitable position spaced from the openings 170, 180 at which the welding can join the inner and outer links 152, 162 together—while the alignment rod is in place. The alignment rod may be removed after welding. In some embodiments, after welding has taken place, the links 142 (whether separately or as assembled as the backbone 140), may be tumbled, electropolished, or otherwise treated (e.g., buffed or smoothed).

The pull wires 121, 122, 123, 124 may be inserted through the channels defined by the inner guide links 157 and the inner pull ring 159. In some instances, the pull wires 121, 122, 123, 124 may be inserted through the guide channels prior to welding. In other instances, this may take place after welding. In various embodiments, the distal tips of the pull wires 121, 122, 123, 124 and/or the connector wires 125, 126 (see FIG. 2A) may be welded to the pull ring 149.

With reference again to FIG. 1, the shaft 104 may be assembled and the backbone 140 incorporated into the bending section 106 in any suitable manner. For example, in some embodiments, the outer sleeve 105 can extend continuously over the backbone 140.

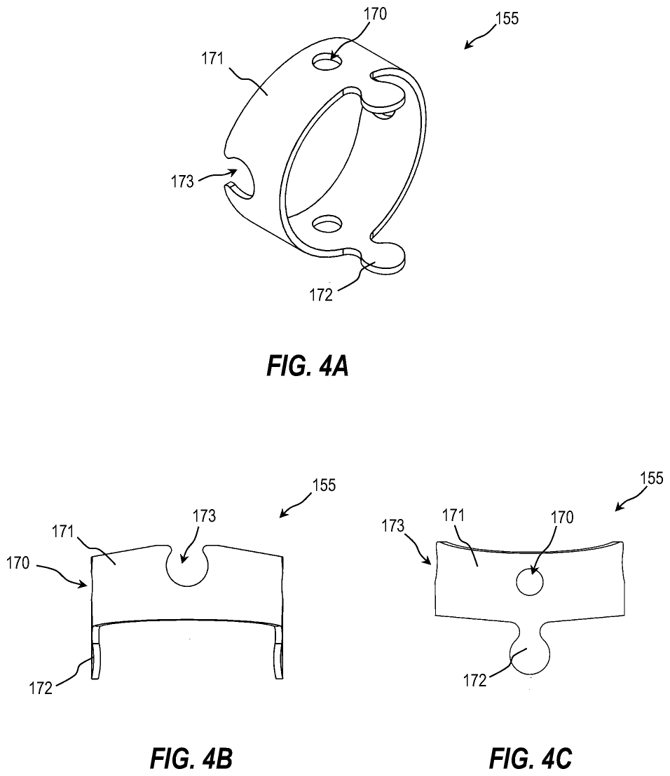

FIGS. 4A-8C depict the various link segments of the illustrated embodiment in further detail.

With reference to FIGS. 4A-4C, in some embodiments, the inner basic link 155 can include a ring or body 171. The body 171 can be formed, in some instances, as a substantially cylindrical ring. The proximal and distal edges or faces of the body 171 may include coupling interfaces for rotatable connection to adjacent inner links. In particular, in the illustrated embodiment, a pair of rounded protrusions or pivot members 172 extend distally from a distal face of the body 171, or stated otherwise, project distally and outwardly from the body 171. In the illustrated embodiment, the pair of pivot members 172 are diametrically opposite one another. Further, a pair of recesses or sockets 173 extend distally from a proximal face of the body 171, or stated otherwise, extend distally and inwardly into the body 171. In the illustrated embodiment the pair of sockets 173 are diametrically opposite one another and are angularly offset from the pair of pivot members 172 by 90 degrees.

As previously noted, the inner basic link 155 can include one or more alignment openings 170. In the illustrated embodiment, the inner basic link 155 includes two alignment openings 170 that are diametrically opposite one another. In the illustrated embodiment, the alignment openings 170 are longitudinally aligned with the pivot members 172.

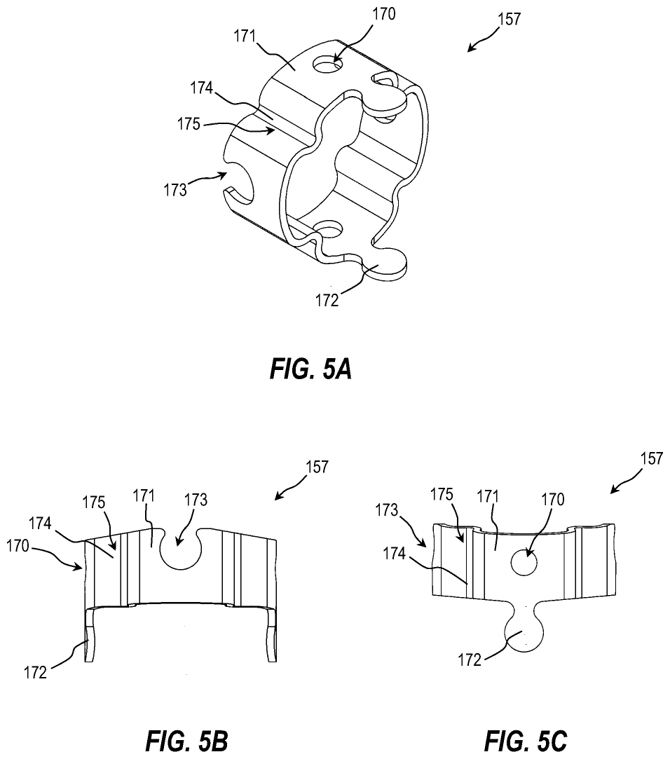

With reference to FIGS. 5A-5C, in certain embodiments, the inner guide link 157 can substantially resemble the inner basic link 155, and can include a body 171, a pair of pivot members 172, a pair of sockets 173, and a pair of alignment openings 170. The pivot members 172 and the sockets 173 can be compatible (e.g., complementary to) those of other inner guide links 157 and/or of other inner basic links 155. The inner guide links 157 can additionally include a plurality of indentations or channels 174 that can serve as or define passages 175, pathways, or guides for the passage and retention of guide wires 121, 122, 123, 124. In the illustrated embodiment, each channel 174 is formed as an inwardly projecting longitudinally elongated indentation. In the illustrated embodiment, the guide link 157 includes four channels 174—one for each guide wire—and each channel 174 is angularly offset from each adjacent channel 174 by 90 degrees. Further, in the illustrated embodiment, the channels 174 are angularly offset from the pivot members 172 and the sockets 173 by 45 degrees.

In other embodiments, different angular offsets of the various features are present. For example, in some instances, the channels 174 may be angularly offset from the pivot members 172 by a greater or lesser amount than they are angularly offset from the sockets. In other or further embodiments, the sockets 173 may not be diametrically opposed relative to each other and/or the pivot members 172 may not be diametrically opposed relative to each other.

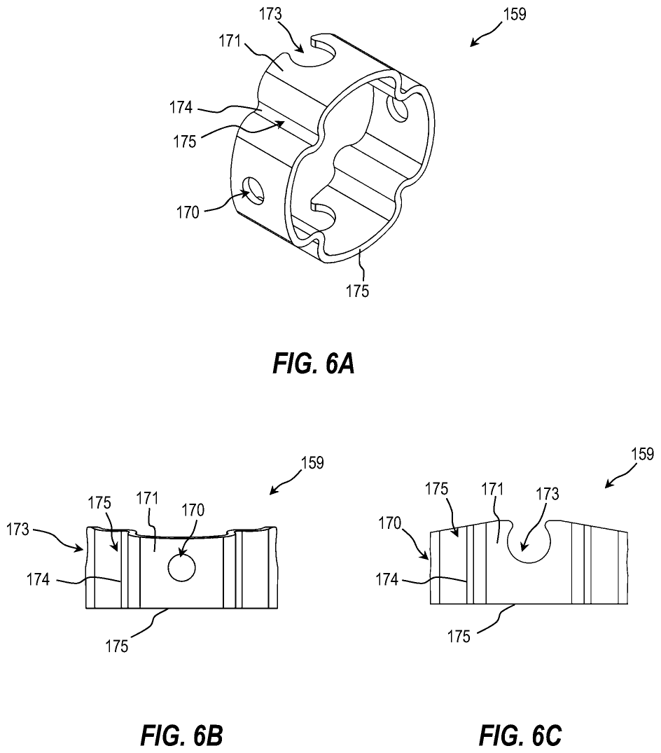

With reference to FIGS. 6A-6C, in certain embodiments, the inner pull ring 159 can substantially resemble the inner guide link 157, and can include a body 171, a pair of sockets 173, four guide wire channels 174, and a pair of alignment openings 170. The sockets 173 of the inner pull ring 159 can be compatible (e.g., complementary to) the pivot members 172 of the inner guide links 157 and/or those of the inner basic links 155. Unlike other inner links, however, the inner pull ring 159 does not include distally projecting pivot members. In the illustrated embodiment, the inner pull ring 159 includes a distal face 175 that is substantially planar, or stated otherwise, is devoid of distal projections or proximal recesses.

As shown in FIG. 3, and as can be appreciated from FIGS. 4B, 4C, 5B, 5C, 6B, and 6C a chain of inner links 152 (e.g., one or more of the inner basic links 155, inner guide links 157, or inner pull ring 159) can include the pivot members 172 of proximally positioned inner links 152 received within the sockets 173 of distally positioned links 152, with each link rotated or angularly offset from adjacent links by 90 degrees. The pivot member 172/socket 173 interfaces can permit rotation of adjacent links relative to each other. Each of the links 152 is rotatable about an axis that passes through the center of a pair of pivot members 172 associated therewith. For example, with reference to FIGS. 4A-4C, each inner basic link 155 is rotatable about an axis that extends through the center of each of the rounded pivot members 172. Likewise, each inner basic link 155 is rotatable about an axis that extends through the center of each of the rounded sockets 173, as these sockets 173 will receive rounded pivot members 172 of an adjacent inner link therein. Thus, each inner basic link 155 is rotatable about two separate axes of rotation. The axes of rotation or longitudinally spaced from one another (e.g., one is proximally positioned, while the other is distally positioned). Further, the axes of rotation are rotationally offset from one another by 90 degrees. Similarly, with reference to FIGS. 5A-5C, each inner guide link 157 is rotatable about two separate axes of rotation, such as just described. With reference to FIGS. 6A-6C, the pull ring 159 is rotatable about a single axis of rotation that extends through the center of the two sockets 173, which sockets can be coupled with a pair of pivot members 172 from either an inner basic link 155 or an inner guide link 157.

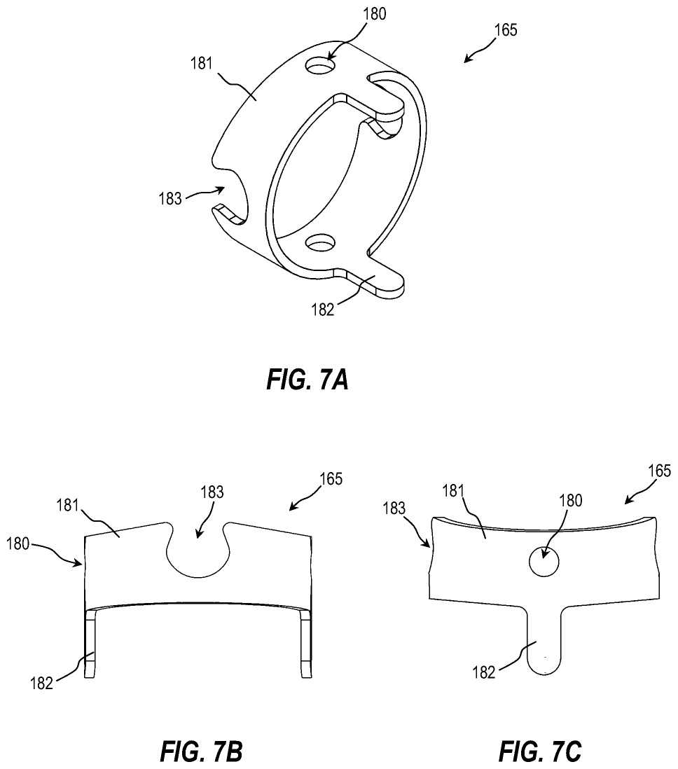

With reference to FIGS. 7A-7C, in some embodiments, the outer basic link 165 can include a ring or body 181. The body 181 can be formed, in some instances, as a substantially cylindrical ring. The proximal and distal edges or faces of the body 181 may include features that permit adjacent outer basic links 165 to rotate relative to each other while locking the coupling interfaces of the inner links 152 in place, as further discussed below. In the illustrated embodiment, a pair of locking protrusions or locking members 182 extend distally from a distal face of the body 181, or stated otherwise, project distally and outwardly from the body 181. In the illustrated embodiment, the pair of locking members 182 are diametrically opposite one another. Further, a pair of recesses 183 extend distally from a proximal face of the body 181, or stated otherwise, extend distally and inwardly into the body 181. In the illustrated embodiment the pair of recesses 183 are diametrically opposite one another and are angularly offset from the pair of locking protrusions 182 by 90 degrees.

As previously noted, the outer basic link 165 can include one or more alignment openings 180. In the illustrated embodiment, the outer basic link 165 includes two alignment openings 180 that are diametrically opposite one another. In the illustrated embodiment, the alignment openings 180 are longitudinally aligned with the locking members 182.

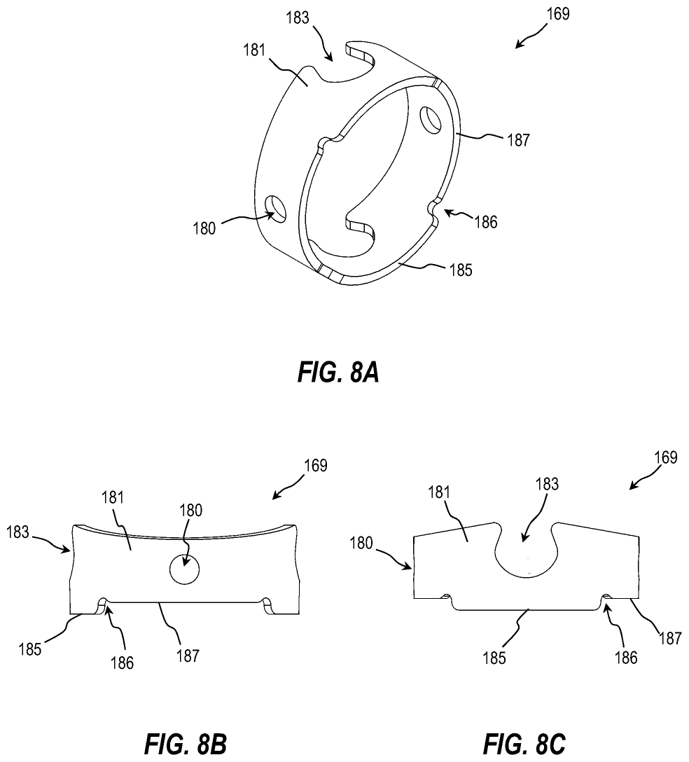

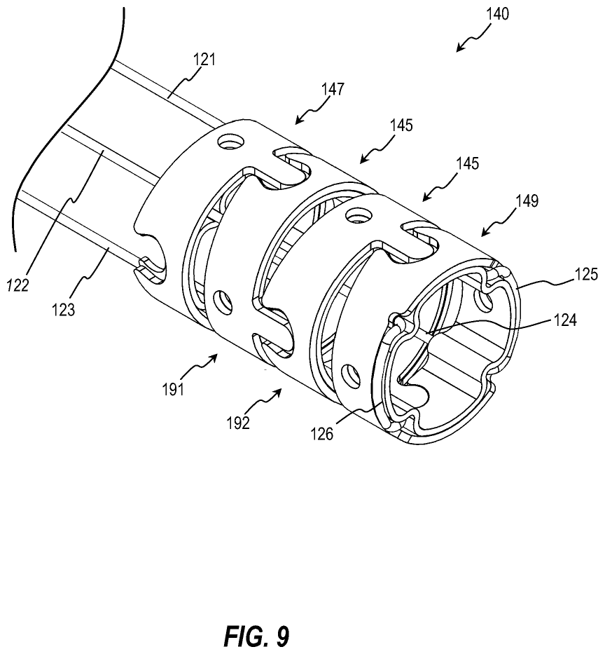

With reference to FIGS. 8A-8C, in certain embodiments, the outer pull ring 169 can substantially resemble the outer basic link 165, and can include a body 181, a pair of recesses 183, and a pair of alignment openings 180. The recesses 183 of the outer pull ring 169 can be compatible with the locking members 182 of the outer basic links 165. Unlike the outer basic link 165, however, the outer pull ring 169 does not include distally projecting locking members. In the illustrated embodiment, the outer pull ring 169 includes two diametrically opposite distal faces 185 that are substantially planar and may be aligned with the distal face 175 of the inner pull ring 159. In some embodiments, the outer pull ring 169 includes a pair of notches 186 at each end of each distal face 185. The notches 186 can be configured to receive distal ends of the pull wires. Extending between pairs of notches 186 are recessed surfaces 187. The pull wires can extend over these surfaces. The recessed surfaces 187 may be recessed by a distance roughly equal to or greater than a thickness of the pull wire. As shown in FIG. 9, when the pull ring 149 is fully assembled and coupled with the pull wires, the distal face of the backbone 140 can be smooth or planar. For example, the distal face 175 of the inner pull ring 159 and/or the distal faces 185 of the outer pull ring 169 can be the distalmost surfaces of the backbone 140, and the distalmost surfaces of the pull wires can be flush therewith or recessed relative thereto.

FIG. 9 depicts the four distalmost links of an illustrative embodiment of the backbone 140. In the illustrated embodiment, this section of the backbone 140 includes a guide link 147, a first basic link 145 positioned distal to the guide link 147, a second basic link 145 positioned distal to the first basic link 145, and a pull ring 149 positioned distal to the second basic link 145, each of which may more generally be referred to as a link 142 (as previously discussed). The backbone 140 is assembled with the guide wires 121, 122, 123, 124. For purposes of the following discussion, the first and second basic links 145 are separately identified as a proximal link 191 and a distal link 192, respectively.

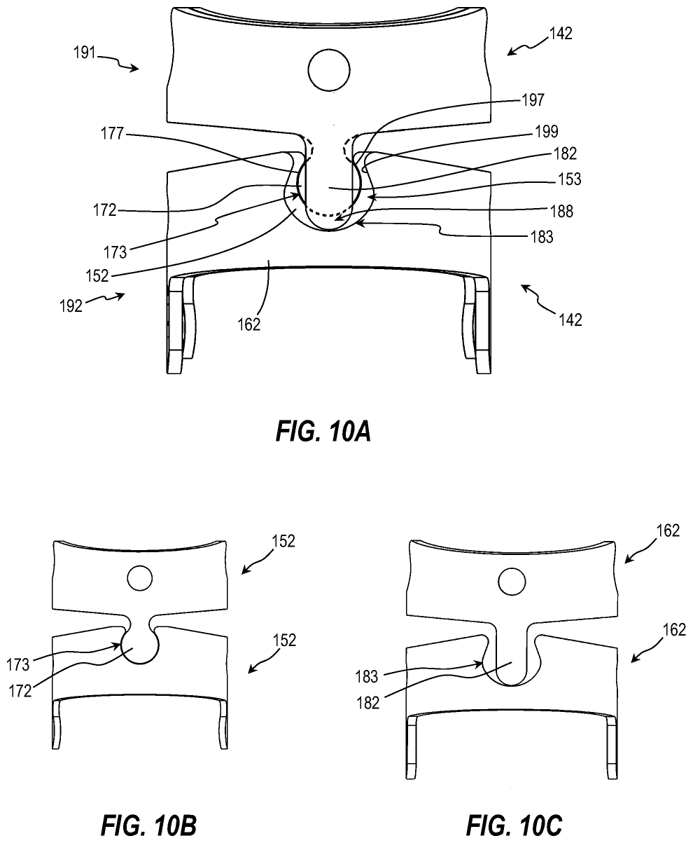

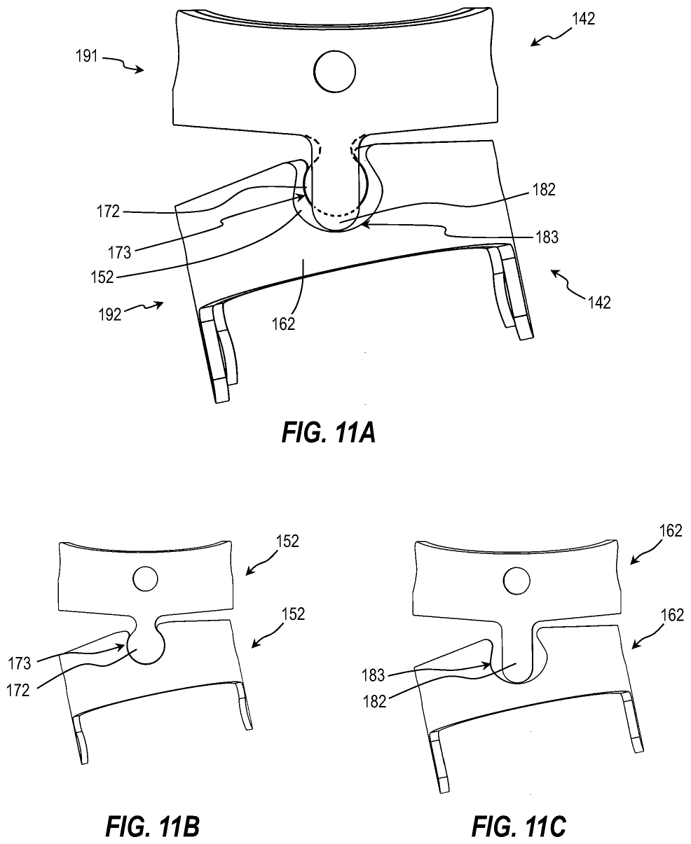

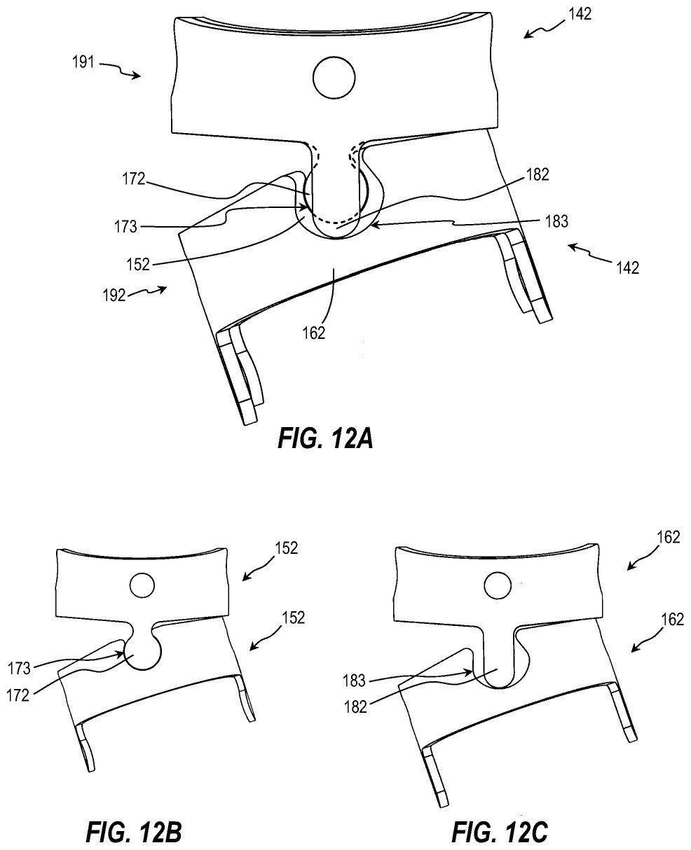

The proximal and distal links 191, 192 are shown in various stages of articulation in FIGS. 10A, 11A, and 12A, and the inner and outer link segments associated with the proximal and distal links 191, 192 are shown in these various stages of articulation in FIGS. 10B, 10C, 11B, 11C, 12B, and 12C. In FIGS. 10A-10C, the proximal and distal links 191, 192 and their associated inner and outer link segments 152, 162 are shown in a straight or unarticulated state. In FIGS. 11A-11C, the proximal and distal links 191, 192 and their associated inner and outer link segments are shown in a first illustrative articulated state. Stated otherwise, the links 191, 192 are shown in a partially articulated or non-fully articulated state, or in an articulated state in which an articulation limit has not been reached. In FIGS. 12A-12C, the proximal and distal links 191, 192 and their associated inner and outer link segments are shown in a second illustrative articulated state. Stated otherwise, the links 191, 192 are shown in a fully articulated state, or in an articulated state in which an articulation limit has been reached.

As can be seen in FIGS. 10A-12C, the locking members 182 of the outer links 162 can permit rotation of the adjacent pair of links 191, 192, while locking the pivot members 172 substantially covered thereby relative to the sockets 173 within which those pivot members 172 are seated. In the illustrated embodiment, the recesses 183 of the outer links 162 are larger than the sockets 173 of the inner links 152, thereby leaving an outer surface of the inner link 152 exposed. A distal tip of each locking member 182 can be positioned over this exposed portion of the outer surface of the inner socket 152 and can slide or pass over the same as the links 191, 192 are rotated. In some instances, the diametrically opposite orientation of the locking members 182 can limit or prevent relative lateral displacement of the proximal inner link 152 (e.g., the link having the pivot members 172) relative to the distal inner link 152 (e.g., the link having the sockets 173 within which the pivot members 172 rotate), thereby maintaining engagement of the pivot members 172 within the sockets 173. It may be said that the assembled links 142 are self-locking.

Stated otherwise, once adjacent pairs of links 142 are fully assembled together, they are locked together in a pivotable coupling. The locking members 182 of the outer links 162 permit the pivot members 172 of the inner links 152 to rotate within their respective sockets 173 while ensuring the pivot members 172 remain engaged with their respective socket members 173. In particular, while each socket 173 permits rotation of its associated pivot member 172 therein, the socket 173 also encompass a sufficient portion of the outer perimeter of the pivot member 172 and/or includes a narrowed or necked region to prevent the pivot member 172 from being pulled or otherwise moved or advanced outwardly from the socket member 173 in a longitudinal direction (e.g., moved out of the socket member 173 in a generally proximal direction, in FIG. 12). Moreover, the distal ends of the locking members 182 are positioned at opposite sides of the inner link 152 so as to prevent lateral movement of one link 142 relative to another link 142 in an amount sufficient to laterally disengage the pivot members 172 from their sockets 173. That is, the distal ends of the locking members 182 prevent relative lateral movements of the links in an amount sufficient to laterally disengage the links 142. The locking members 182 prevent each pivot member 172 from moving laterally inwardly or laterally outwardly in an amount sufficient to disengage from its respective socket 173.

Stated in yet another manner, in each pairing of proximal and distal links 191, 192, the locking members 182 of the proximal outer link 162 can interact with the exposed outer surface of the distal inner link 152 to prevent lateral displacement of the links 191, 192 relative to one another. This lateral locking can ensure that the pivot member 172 of the proximal inner link 152 remains positioned within the socket 173 of the distal inner link 152. With the pivot member 172 and the socket 173 thus maintained in a coupled arrangement, the geometry of the pivot 172 and the socket 173 further prevents the inner links 152 from being pulled apart from each other in a longitudinal direction, while nevertheless permitting rotation of the inner links relative to each other.

By way of example, with reference to FIGS. 10A, 11A, and 12A, it can be seen that at the side of the links 191, 192 that is visible in the drawings, an arc-shaped or, in this particular embodiment, horseshoe-shaped portion of an outer surface of the distal inner link 152 is visible, being exposed by the recess 183 of the distal outer link 162. In particular, the recess 183 of the distal outer link 162 is larger than the socket 173 of the distal inner link 152, which leaves the horseshoe-shaped outer surface of the distal outer link 162 exposed. While the pivot member 172 of the proximal inner link 152 is rotatably secured within the socket 173 of the distal inner link 152, a distal end of the locking member 182 of the proximal outer link 162 is positioned over the horseshoe-shaped portion of the outer surface of the distal inner link 152. In the illustrated embodiment, an identical arrangement is found at a diametrically opposite side of the links 191, 192, or stated otherwise, at the opposite side of the links 191, 192 that is not visible in these drawings.

In the arrangement just described, the locking members 182 can maintain the links 191, 192 in a coupled state, counteracting laterally displacing forces that might otherwise tend to separate the links 191, 192 from each other. For example, if an inwardly directed force (i.e., a force directed away from the viewer in the illustrated view) were to be exerted on the proximal link 191 while the distal link 192 were held in place, in the absence of the illustrated locking member 182, the force would tend to slide the pivot member 172 of the proximal inner link 152 out of the socket 173 of the distal inner link 152. Such a situation can be envisaged by reference to, e.g., FIG. 10B. Due to the presence of the locking member 182, however, the lateral displacement force applied to the proximal link 191 is counteracted by the interfacing of the distal tip of the locking member 182 with the exposed outer surface of the distal inner link 152. That is, the portion of the locking member 182 that overlies the exposed portion of the inner link 152 acts to prevent lateral movement of the proximal link 191 relative to the distal link 192 in a first direction.

Similarly, if an oppositely directed lateral displacement force (i.e., a force directed toward the viewer in the illustrated view) were to be exerted on the proximal link 191 while the distal link 192 were held in place, this force would be counteracted by the interfacing of the distal tip of the non-viewable locking member 182 at the opposite side of the proximal link 191 with the exposed outer surface of the distal inner link 152 that is likewise at the opposite side of the distal link 192. That is, the portion of the locking member 182 that overlies the exposed portion of the inner link 152 at the opposite, nonviewable side acts to prevent lateral movement of the proximal link 191 relative to the distal link 192 in a second direction that is opposite the first direction. The locking members 182 can inhibit or prevent disconnection of the links 191, 192—e.g., can inhibit or prevent relative lateral movement of the links 191, 192 relative to each other. The locking members 182 can counteract lateral displacement forces, interacting with the underlying inner links 152, and can maintain the pivot members 172 in engaged relation with their respective sockets 173. Thus engaged, the pivot members 172 and sockets 173 can inhibit or prevent disconnection of the links 191, 192—e.g., can inhibit or prevent longitudinal displacement of the links 191, 192 relative to each other—while permitting the links 191, 192 to rotate relative to each other.

In some embodiments, a proximal portion of each locking member 182 may similarly inhibit or prevent relative lateral movement of the links 191, 192. For example, in the illustrated embodiment, two small portions of the inner surface located at an upper end of each locking member 182 defined by the proximal outer link 162 are exposed due to a narrow necking at a proximal end of the associated pivot member 172 of the inner link 152. Each exposed portion may overlie a portion of the horseshoe-shaped exposed outer surface of the distal inner link 152, such as when the links 191, 192 are rotated relative to each other (see FIGS. 11A and 12A). Interaction of the exposed inner and outer surfaces of the outer and inner links 162, 152, respectively, can inhibit or prevent, or can contribute to the inhibition or prevention, of lateral movement in manners such as previously discussed.

With reference to FIG. 10A, the pivot member 172 can include a peripheral edge 177 that defines the outer contour of the pivot member 172. That is, an outline of the pivot member 172 can be defined by its peripheral edge 177. The peripheral edge 177, identified in FIG. 10A, is shown in both solid and broke lines in each of FIGS. 10A, 11A, and 12A. Those portions of the peripheral edge 177 that are covered by the locking member 182 are shown in broken lines, whereas those portions of the peripheral edge 177 that are not covered by the locking member 182 are shown in solid lines. In the illustrated embodiment, the locking member 182 overlies only a portion of the pivot member 172. In other embodiments (see, e.g., FIG. 35), the locking member 182 may overlie an entirety of the pivot member 172.

With continued reference to FIG. 10A, a peripheral edge 197 of the socket 173 of one inner link 152 may be substantially complementary to the peripheral edge 177 of the pivot member 172 of an adjacent inner link 152. For a given link 142, a peripheral edge 199 of a recess 183 of the outer link 162 may be spaced from the peripheral edge 197 of the socket 173. This may leave exposed a portion of the outer surface of the inner link 152 with which the locking member 182 may interface, as previously discussed. This exposed region, which is substantially arc- or horseshoe-shaped in the illustrated embodiment, may be referred to as a locking surface 153 of the inner link 152. A portion of the locking member 182 that extends beyond the peripheral edge 177 of the locking member 182 may be referred to as a locking section 188 of the locking member 182. As can be seen in FIG. 10A, the locking section 188 of one link 142 (e.g., the top link in the illustrated view) can overlie the locking surface 153 of an adjacent link 152 (e.g., the bottom link in the illustrated view). In manners such as previously discussed, the locking section 188 of a first link 142 can interfere or otherwise interact with the locking surface 153 of a connected second link 142 to inhibit or prevent relative lateral movement of the connected first and second links 142, e.g., in a first direction. For example, this interaction can prevent the upper link 142 from moving laterally relative to the lower link 142 in a direction away from the viewer in FIG. 10A. Moreover, in the illustrated embodiment, another locking section 188/locking surface 153 coupling is present at a diametrically opposed side of the first and second links 142 (i.e., at the side of the links 142 facing away from the viewer in FIG. 10A), which also can inhibit or prevent relative lateral movement of the first and second links 142, e.g., in a second direction. For example, this interaction can prevent the upper link 142 from moving laterally relative to the lower link 142 in a direction toward the viewer in FIG. 10A. The pair of opposing locking sections 188 thus can cooperate with the corresponding pair of locking surfaces 153 to fully inhibit or prevent relative lateral movement of the first and second links 142, which can maintain the pivot members 172 coupled within respective sockets 173 at opposite sides of the links 142.

With continued reference to FIG. 10, in the illustrated embodiment, the locking member 182 is substantially tongue-shaped, or stated otherwise, includes a narrow extension with substantially parallel sides and a rounded end cap that joins the parallel sides. The parallel sides define a width that is smaller than a diameter of the pivot member 172 that the locking member 182 overlies. Stated otherwise, the locking member 182 is narrower than a maximum width of the pivot member 172. The illustrated locking member 182 does not fully cover the pivot member 172, or stated otherwise, overlies only a portion of the pivot member 172.

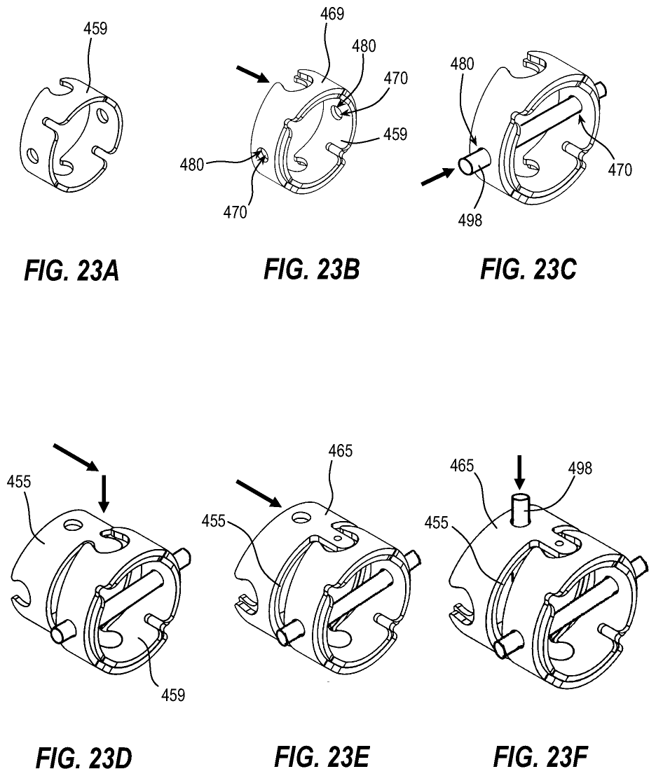

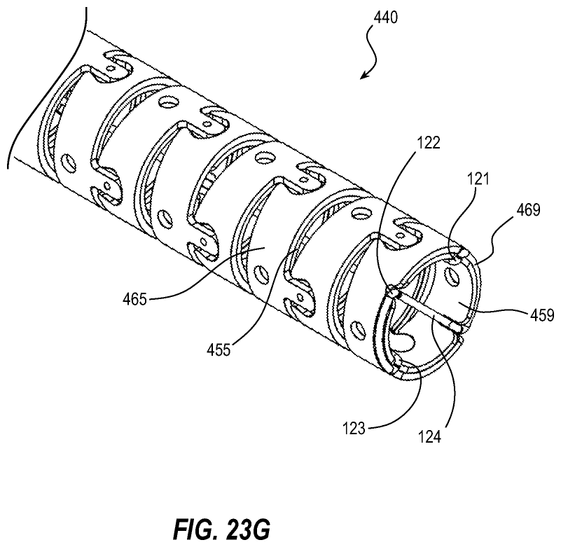

In some embodiments, it can be advantageous for the locking member 182 of one link 142 to include a locking section 188 that overlies only a portion of the locking surface 153 of an adjacent, connected link 142. In some instances, such an arrangement can reduce an amount of friction or resistance to rotation of one link 142 relative to the other. This may, for example, in some instances, be desirable in certain endoscopic arrangements in which articulation of a bending section is preferably achieved with lower forces on pull wires. In other or further instances, such an arrangement may distance the periphery of the locking member 182 of one link 142 from the edges of the socket 172 of the adjacent link 142. In some instances, flexural stresses or other changes in geometry of the socket 172 that may arise as one link 142 rotates relative to an adjacent link 142 might tend to urge the locking member 182 and its associated pivot member 172 outwardly relative to the socket 173, wherein greater coverage of the locking surface 153 is present. A narrower locking member 182 may ameliorate or avoid such interactions, which could tend to disengage the pivot member 172 from the socket 173. In some embodiments, tongue-shaped or narrow locking members 182 may facilitate assembly of a backbone 140. For example, in some instances, the narrow locking members 182 may be simultaneously advanced through the narrow neck regions defined by the recesses 183 as one outer link is advanced toward another (see, e.g., FIGS. 23E and 23G).

In other embodiments, the locking section 188 may overlie significantly more of the locking surface 153. For example, in some embodiments (see, e.g., FIG. 35), the locking member 182 may overlie an entirety of a pivot member 172 and the locking section 188 of the locking member 182 may substantially encompass the peripheral edge 177 of the pivot member 172. In some embodiments, such an arrangement can provide relatively greater resistance to lateral movement of one link 142 relative to another.

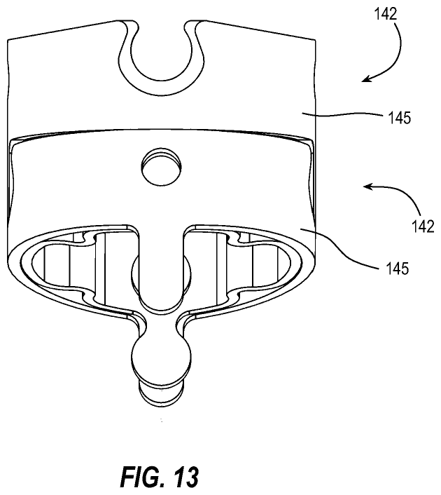

In various embodiments, the geometries of the links 142 can be readily altered to achieve a desired bend configuration of the backbone 140. For example, in some instances, when all links 142 of a backbone 140 are in a fully articulated configuration (in a single plane), the backbone 140 may define a semi-circular profile. A minimum radius of curvature of the fully articulated backbone 140 can be adjusted by permitting a greater amount of relative rotation between adjacent links before contact such as that depicted in FIGS. 12A and 13 is made. Thus, for example, a greater amount of material may be removed from adjacent links to create a larger gap between the links when they are in an unarticulated configuration (such as shown in FIG. 10A). This can allow a greater amount of relative rotation, which can yield a smaller minimum radius of curvature for the fully articulated backbone 140. Likewise, a larger minimum radius of curvature can be achieved by reducing the gap size.

In other or further instances, other profiles for a fully articulated backbone 140 are achievable by varying gap sizes or other geometric dimensions along a length of the backbone 140. For example, in some instances, a substantially J-shaped backbone 140 (fully articulated in a single plane in one direction) can be achieved with distally positioned links that yield a small radius of curvature (e.g., a more aggressive bend) and proximal links that yield a much larger radius of curvature (e.g., a less aggressive bend). Any other suitable configuration is contemplated. Moreover, similar adjustments and selections may be made to achieve the same and/or different configurations and/or radii of curvature when the backbone 140 is fully articulated in a second direction along the first plane and/or along one or two directions along a second plane that is orthogonal to the first plane. Other articulation configurations are also readily achievable.

In some embodiments, the backbone 140 can be lockable in a predetermined or prespecified shape. For example, in some embodiments, the backbone 140 may be used in a steerable catheter suitable for use in mitral valve delivery. The links of the backbone may be lockable in a shape suitable for such delivery.

FIG. 13 depicts another pair of adjacent links 142, with the upper link 142 being a basic link 145 and the lower link 142 being a guide link 147. The links 142 are rotated to an articulation limit. Stated otherwise, a maximum angular displacement of one link 142 relative to the other has been reached. Contact between a distal face of the proximal link 142 and a proximal face of the distal link 142 can delimit the rotational movement. In some embodiments, the faces of the adjacent links 142 may be saddle shaped. In some instances, such as saddle shape can allow the adjacent links to contact one another along a line, rather than a single point, which can stabilize a curvature of the backbone when the articulation limit has been reached.

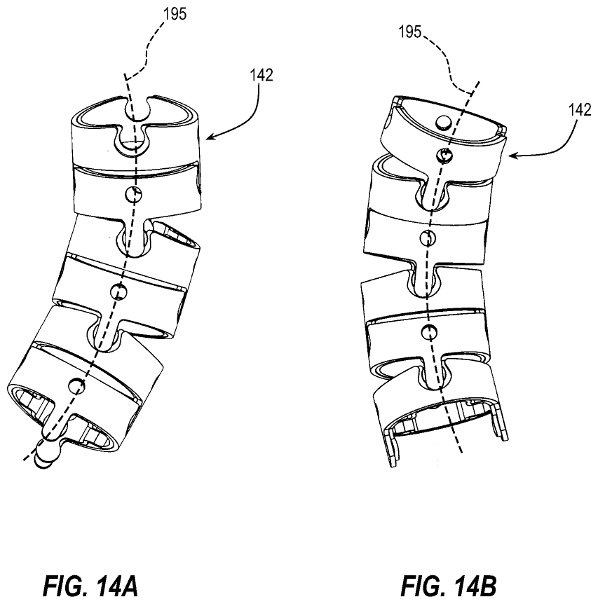

FIG. 14A is a front perspective view of six interconnected links shown in an articulated state, wherein three distinct link doublets are shown, with each doublet having rotated in unison in a first dimension to define a first arc 194. FIG. 14B is a side perspective view of the six interconnected links of FIG. 14A shown in the articulated state, the view being rotated approximately 90 degrees counterclockwise (in a proximal-to-distal reference frame) about a longitudinal axis of the backbone relative to the view depicted in FIG. 14A, wherein two distinct link doublets are shown, with each doublet having rotated in unison in a second dimension to define a second arc 195.

As can be appreciated from the present disclosure, including the depictions in FIGS. 14A and 14B, in some embodiments, adjacent links may rotate in unison or be locked relative to one another in a first dimension, yet may rotate freely relative to each other in another dimension. The first and second dimensions may be angularly offset by 90 degrees. For example, it may be said that the three sets of link doublets that bend in unison in a first dimension split up such that the links rotate relative to each other in the second dimension.

Other embodiments can include one or more reversals of components or features. For example, in some embodiments, one or more pivot members 172 and sockets 173 may be reversed and/or one or more lock members 182 and recesses 183 may be reversed. In other or further embodiments, one or more sets of outer links 162 may define pivot members and sockets while one or more corresponding sets of inner links may define lock members and recesses. In some instances, it can be advantageous for the lock members and recesses to be defined by outer link members, as the lock members may be able to move more freely relative an outer surface of an inner link member when rotated than they might be relative to an inner surface of an outer link member.

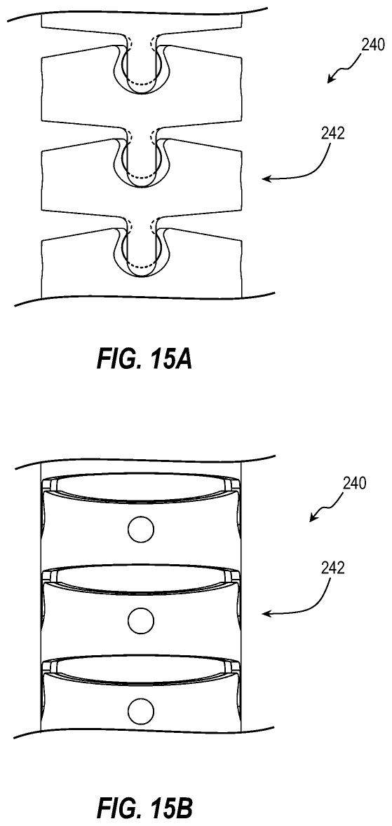

Some embodiments may be suitable for articulation in a single plane, or can achieve two-directional articulation, rather than four-directional articulation in two planes. For example, FIG. 15A is a front elevation view of another embodiment of an articulable backbone 240 that includes a plurality of interconnected links 242 that are configured for two-directional articulation, such as in opposite directions in a single plane. FIG. 15B is a side elevation view of the backbone 240 that is rotationally offset about a longitudinal axis of the backbone 240 by 90 degrees relative to the view depicted in FIG. 15A. The links 242 may be similar to any of the links 142 (e.g., the links 145, 147, 149) described above. The links 242 may be configured for use with a single pair of pull wires, and may only define two lines of pull wire channels at an interior thereof.

In still other embodiments, articulation may be achieved in a single direction only. For example, end faces of adjacent links 242 may abut one another on one side of the links 242 when in a straight configuration (e.g., along the left side in a view such as that of FIG. 15A) so as to prevent bending along that side, whereas an opposite side (e.g., the right side in a view such as that of FIG. 15A) can include gaps between adjacent faces to permit articulation along that side of the links 242.

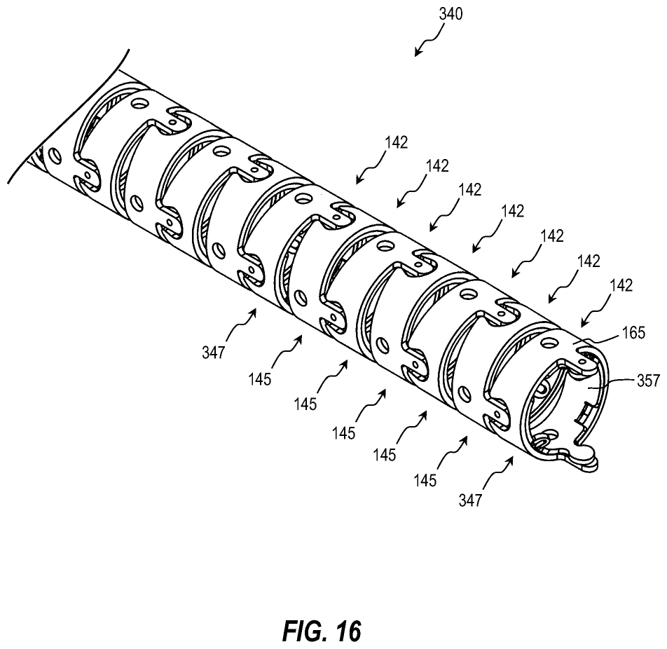

FIG. 16 is a perspective view of another embodiment of an articulable backbone 340 that is compatible with the bending section of, for example, an endoscope, such as the endoscope 100 discussed above. The backbone 340 can resemble the backbone 140 in many respects. In some embodiments, the backbone 340 includes numerous components similar or identical to those of the backbone 140. In the illustrated embodiment, the backbone 340 includes a series or chain of interconnected links 142 that can be rotated relative to one another in manners such as previously disclosed. Some of the links 142 are basic links 145, which can be substantially the same as or identical to the basic links 145 discussed above. Some of the links 142 can comprise another embodiment of guide links 347, which can differ somewhat from the guide links 147 previously described, as discussed further below. Although not shown in FIG. 16, the backbone 340 can include a pull ring or terminal link 149, such as described above, at a distal end thereof.

With continued reference to FIG. 16, in the illustrated embodiment, relatively fewer guide links 347 are used, as compared with the backbone 140. In particular, adjacent pairs of guide links 347 are separated by five basic links 145, as compared with a separation of only two basic links 145 between adjacent pairs of guide links 147 in the backbone 140 (see FIG. 2A). As previously discussed, any suitable number or pattern of guide links 347 and basic links 145 are contemplated. For example, in other embodiments, only guide links 347 may be used, along with a terminal link 149, and basic links 145 may be omitted.

As shown in FIG. 16, in some embodiments, each guide link 347 can include a basic outer link 165, such as previously described. Each guide link 347 can include an embodiment of an inner guide link 357 that can differ somewhat from the inner guide link 157 previously described.

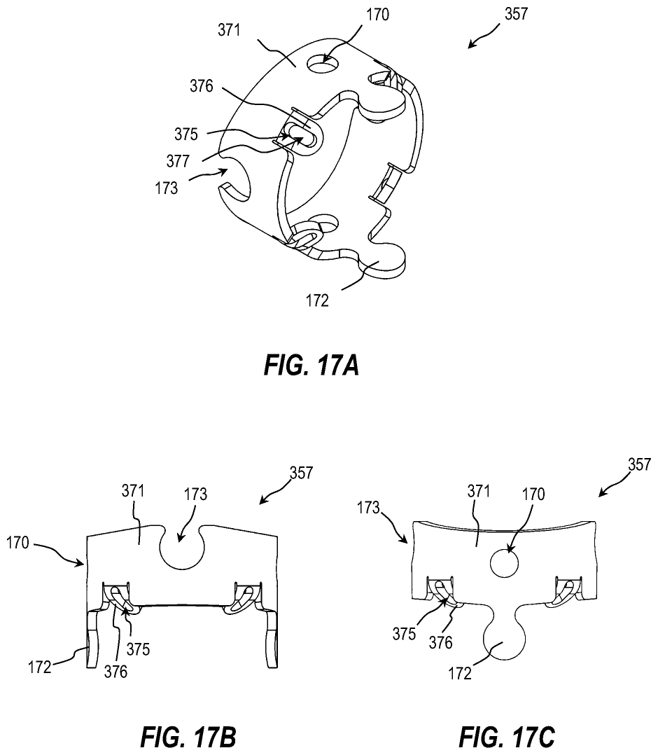

With reference to FIGS. 17A-17C, the inner guide link 357 can include a body or ring 371 that does not include longitudinal indentations or channels as guides. Rather, the inner guide link 357 includes a plurality of rings, loops, or eyelets 376, which may be laser cut and bent inward. In some instances, laser-cut and bent eyelets 376 may be faster and/or cheaper to manufacture and/or may facilitate assembly of an endoscope or other elongated instrument into which the backbone 340 is incorporated.

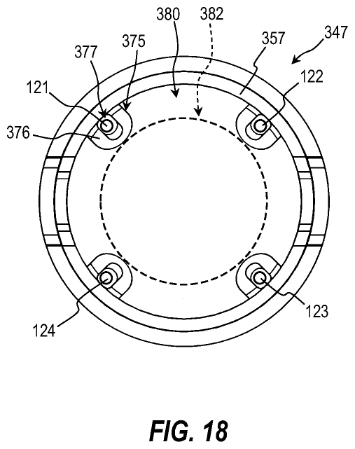

The eyelets 376 can include openings 377 through which the pull wires pass. The eyelets 376 can function as guides 375 or retainers for the pull wires. In the illustrated embodiment, the inner guide link 357 includes four eyelets 376, each angularly separated from adjacent eyelets by 90 degrees. In the illustrated embodiment, each eyelet 376 is configured to receive therethrough a separate pull wire 121, 122, 123, 124, as shown in FIG. 18.

As with the inner guide link 157, the inner guide link 357 can include pairs of alignment openings 170, pivot members 172, and sockets 173. The inner guide links 357 can function substantially as described above with respect to the inner guide links 157.

With reference to FIG. 18, the guide link 347 can define a lumen 380 through which any desirable items may pass. For example, in various embodiments, the lumen 380 may receive therein one or more of a working channel through which any suitable instrument, implement, or accessory may pass; power and/or communication lines, such as electrical wiring or cables (e.g., for a camera and/or one or more LEDs mounted at a distal end of an endoscope); light guides (e.g., for alternative lighting arrangements); one or more water lines; one or more air lines; etc.

In various embodiments, it may be desirable for a substantial portion of the lumen 380 to remain unencumbered. This can permit all necessary or desired components to pass through the lumen 380 for the specific instrument into which the backbone 340 is incorporated, while maintaining a low-profile. For example, in some instances, it can be desirable for an outer diameter of the elongated shaft 140 of an endoscope 100 (see FIG. 1) to be as small as possible without impacting functionality. In some instances, one or more tubes may extend through the lumen 380. In some instances, the largest possible space a cylindrical tube may occupy within the lumen 380 may be delimited by the eyelets 376. For example, in the illustrated embodiment, an outer boundary of a cylindrical accessory space 382 is shown. In cross-section, this circular space has a diameter that is smaller than an inner diameter of the inner guide link 357. In particular, in the illustrated embodiment, a maximum diameter of the cylindrical accessory space 382 is smaller than the inner diameter of the inner guide link 357 by a distance equal to twice the length of an eyelet 376 (where all eyelets 376 are the same size). Stated otherwise, the maximum diameter of the cylindrical accessory space 382 is smaller than the inner diameter of the inner guide link 357 by a distance equal to the maximum combined length of oppositely directed, or diametrically opposite, eyelet pairs. If a cylindrical tube were to be advanced through the guide link 347, a maximum diameter of the tube could correspond with the diameter of the cylindrical accessory space 382.

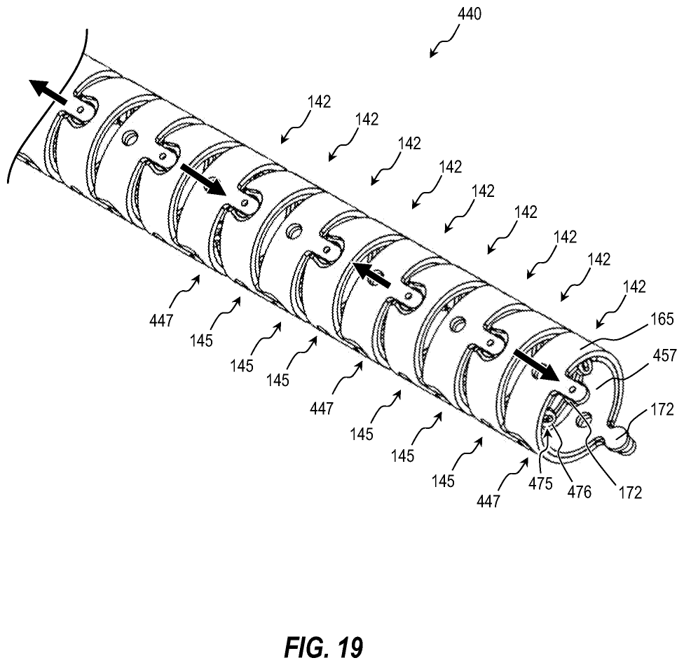

FIG. 19 is a perspective view of another embodiment of an articulable backbone 440 that is compatible with the bending section of, for example, an endoscope, such as the endoscope 100 discussed above. The backbone 440 can resemble the backbones 140, 340 in many respects. In some embodiments, the backbone 440 includes numerous components similar or identical to those of one or more of the backbones 140, 340. In the illustrated embodiment, the backbone 440 includes a series or chain of interconnected links 142 that can be rotated relative to one another in manners such as previously disclosed. Some of the links 142 are basic links 145, which can be substantially the same as or identical to the basic links 145 discussed above. Some of the links 142 can comprise another embodiment of guide links 447, which can differ somewhat from the guide links 147, 347 in manners such as described hereafter. For example, as further discussed below, the guide links 447 may allow for a relatively larger cylindrical accessory space, as compared with certain embodiments of the guide links 147, 347.

With continued reference to FIG. 19, in the illustrated embodiment, each guide link 447 includes an outer basic link 165 and an inner guide link 457. Each inner guide link 457 includes two guides 475 in the form of inwardly directed eyelets 476. The guides 475 flank either side of (e.g., are angularly spaced 45 degrees away from, on either side of) one of the two pivot members 172 defined by the inner guide link 457. The other pivot member 172 is not flanked by guides.

For each pair of adjacent guide links 447, the guide links 447 are separated from one another by three intervening basic links 145. Moreover, as depicted by bold arrows that generally point in a direction in which the eyelets 476 of a given guide link 447 are directed (i.e., either substantially upward and leftward or substantially downward and rightward, in the illustrated view), each guide link 447 is rotated 180 degrees from adjacent guide links 447. Stated otherwise, the guide links 447 are serially rotated by 180 degrees along a length of the backbone 440. In such an arrangement, the intervening basic links 145 can provide spacing within which a substantially cylindrical tubular region can gradually undulate back and forth along the longitudinal length of the backbone 440.

As previously discussed, any suitable number or pattern of guide links 447 and basic links 145 are contemplated. Any suitable rotational pattern of adjacent guide links 447 is also contemplated.

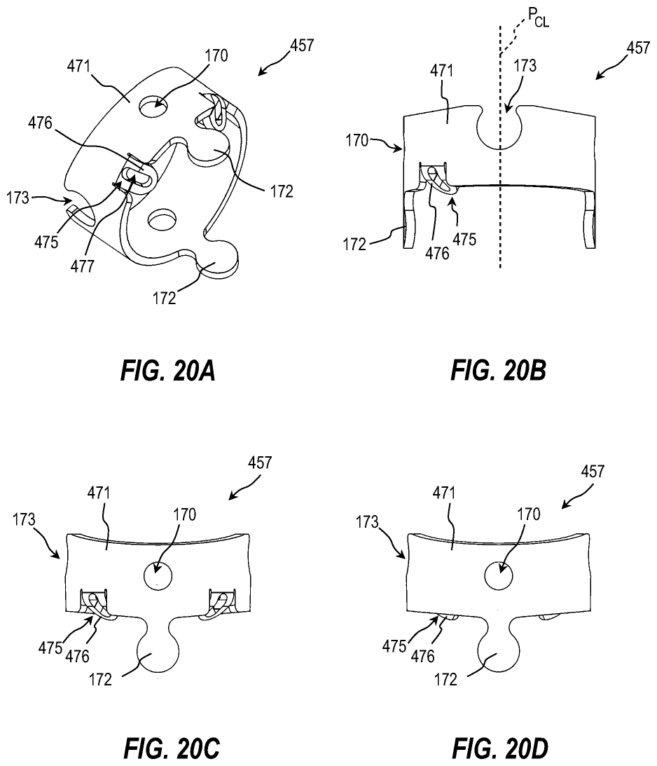

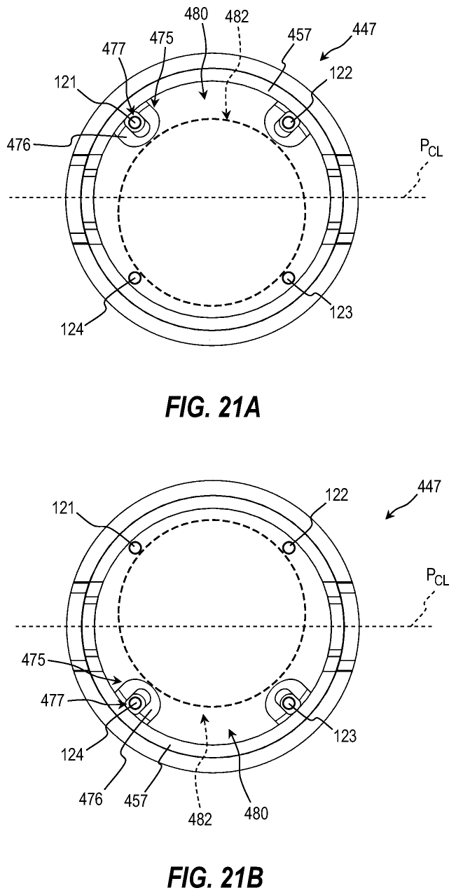

With reference to FIGS. 20A-20D, the inner guide link 457 can include a body or ring 471 that includes a pair of rings, loops, or eyelets 476 that extend from a distal end of the ring 471. As with the eyelets 347, the eyelets 476 may be laser cut and bent inward. The eyelets 476 can include openings 477 through which the pull wires pass. The eyelets 476 can function as guides 475 or retainers for the pull wires. In the illustrated embodiment, the inner guide link 457 includes two eyelets 476, each angularly separated from the adjacent eyelet by 90 degrees (in one direction, and 270 degrees in the opposite direction). In the illustrated embodiment, the eyelets 476 are positioned at opposite sides of a pivot member 172, each being angularly spaced therefrom by 45 degrees. In the illustrated embodiment, the pair of eyelets 476 is configured to receive therethrough two of the pull wires 121, 122, 123, 124, depending on rotational orientation, as shown in FIGS. 21A and 21B. That is, each eyelet 476 is configured to receive therethrough a separate, single one of the pull wires 121, 122, 123, 124.

The inner guide link 457 can include pairs of alignment openings 170, pivot members 172, and sockets 173 such as those disclosed previously. The inner guide links 457 can function substantially as described above with respect to the inner guide links 157, 357. As previously noted, the pair of eyelets 476 can flank one of the two pivot members 172, in the illustrated embodiment. Stated otherwise, in the illustrated embodiment, a central longitudinal plane P_{CL} (FIG. 20B) can bisect the inner guide link 457 along a central longitudinal axis thereof. In the illustrated embodiment, the central longitudinal plane P_{CL} extends between and is equidistant from the opposing pivot members 172. Both of the eyelets 476 are positioned on one side of the central longitudinal plane P_{CL}, while no eyelets 476 are positioned on the other side of the central longitudinal plane P_{CL}.

FIG. 21A depicts a guide link 447 that is rotated such that its eyelets 476 are positioned at a first side of the central longitudinal plane P_{CL}, whereas FIG. 21B depicts a guide link 447 of identical construction that is rotated 180 degrees relative to the guide link 447 in FIG. 21A, such that its eyelets 476 are positioned at a second side of the central longitudinal plane P_{CL} that is opposite the first side. Each guide link 447 can define a lumen 480 such as the lumen 380 previously described. In some instances, the largest possible space a cylindrical tube may occupy within the lumen 480 may be delimited by the eyelets 476 of that guide link 447 and a pair of the pull wires 121, 122, 123, 124 that is positioned outside of those eyelets 476. For example, in each of FIGS. 21A and 21B, an outer boundary of a cylindrical accessory space 482 is shown. In FIG. 21A, the size of this cylindrical accessory space 482 is delimited by the radially innermost tips of two eyelets 476 (which have pull wires 121, 122 positioned therein) and the outer surfaces of two further pull wires 123, 124 that are external to the eyelets 476. In FIG. 21B, which is at a position spaced from the guide link 447 of FIG. 21A, the size of this cylindrical accessory space 482 is delimited by the radially innermost tips of two eyelets 476 (which have pull wires 123, 124 positioned therein) and the outer surfaces of the pull wires 121, 122 that are external to the eyelets 476. In cross-section, this circular space has a diameter that is smaller than an inner diameter of the relevant inner guide link 457. In particular, in the illustrated embodiment, a maximum diameter of the cylindrical accessory space 482 is smaller than the inner diameter of a given inner guide link 457 by a distance equal to the maximum combined length of oppositely directed, or diametrically opposite, eyelet/pull wire pairs. If a cylindrical tube, such as may be used for a working channel, were to be advanced through the guide links 447, a maximum diameter of the tube could correspond with the diameter of the cylindrical accessory space 482.

In FIG. 21A, the cylindrical accessory space 482 is off-centered relative to the central longitudinal plane P_{CL}. In this drawing, the cylindrical accessory space 482 is shifted downward on the page. This off-centering is due to the larger size of the eyelets 476 as compared with the outer diameters of the pull wires 123, 124. Similarly, in FIG. 21B, the cylindrical accessory space 482 is off-centered and shifted upward on the page relative to the central longitudinal plane P_{CL}. Thus, as previously noted, the cylindrical accessory space 482 can gradually undulate back and forth along the length of the backbone 440. The cylindrical accessory space 482 may more accurately be referred to as substantially cylindrical due to these back-and-forth variations along a longitudinal length of the backbone 440. As previously noted, the intervening basic links 145 between adjacent guide links 447 can provide spacing within which this substantially cylindrical tubular region can gradually meander back and forth. In some instances, a flexible tube may fill the substantially cylindrical accessory space 482 and may undulate back and forth relative to the central longitudinal plane P_{CL} along the longitudinal length of the backbone 440.

As previously noted, in the illustrated embodiment, each guide link 447 is separated from an adjacent guide link 447 by three basic links 145, as shown in FIG. 19. Such an arrangement can permit gradual undulation of a guide tube positioned within the backbone 440. In other embodiments, basic links 145 may be omitted. For example, in some embodiments, only guide links 447 may be used, or in further embodiments, only guide links 447 are used other than at the distal and/or proximal ends of the backbone 440, where a pull ring and/or proximal connector may be used, respectively. Such an arrangement can reduce a variety of parts used in manufacturing the backbone 440. Any suitable orientation of the various guide links 447 is contemplated. For example, in some embodiments, a first series (e.g., two in a row, three in a row, etc.) of guide links 447 may be oriented in a first direction, and a subsequent or adjacent second series (e.g., two in a row, three in a row, etc.) of guide links 447 may be rotated at 180 degrees relative to the first series. Multiple series of guide links 447 may follow this pattern. In other embodiments, the guide links 447 may alternate directions, at 180-degree rotations, one after another along a full or partial length of the backbone 440. Other arrangements and configurations are contemplated.

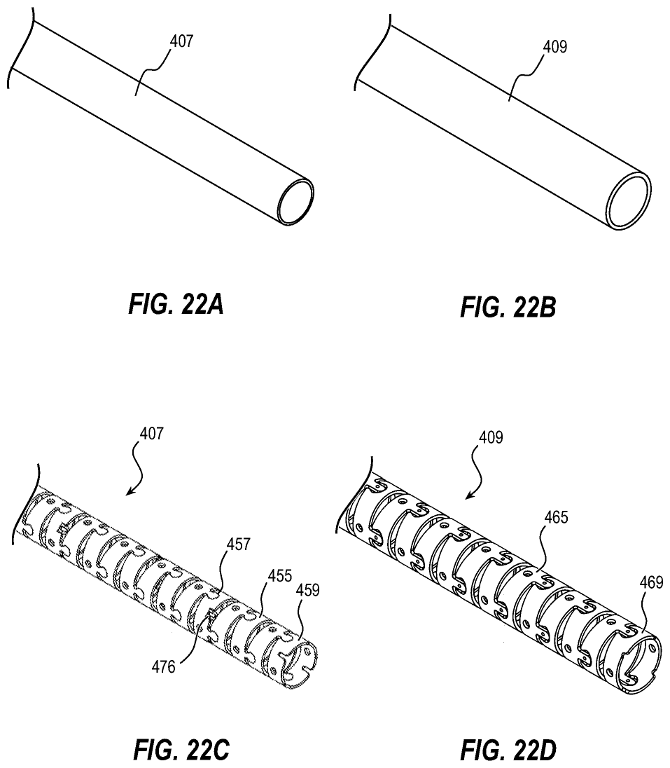

FIGS. 22A-23G depict various stages of an illustrative method of manufacturing a backbone 440. The illustrative method and/or portions thereof can be used with at least some of the embodiments of backbones disclosed herein. The illustrated method depicts the manufacture of an embodiment of the backbone 440.

In FIG. 22A, a preformed tube 407 is provided. The preformed tube 407 can be substantially cylindrical, in some embodiments. The tube 407 can define an inner diameter and an outer diameter. The tube 407 may be formed of a metallic material, in some instances, although other materials are contemplated. For example, in some embodiments, the tube 407 may be plastic. In various embodiments, the tube 407 may be hypotube, such as a stainless steel hypotube. The tube 407 may be rigid.

In FIG. 22B, a further preformed tube 409 is provided. As with the tube 407, the tube 409 can be substantially cylindrical, in some embodiments. The tube 409 can define an inner diameter and an outer diameter. The tube 409 may be formed of a metallic material, in some instances, although other materials are contemplated. For example, in some embodiments, the tube 409 may be plastic. In various embodiments, the tube 409 may be hypotube, such as a stainless steel hypotube. The tube 409 may be rigid.

In some embodiments, the inner diameter of the tube 409 is only slightly larger than the outer diameter of the tube 407. For example, the tube 407 may be configured to be positioned inside of the tube 409, such as by sliding the inner tube 407 into the outer tube 409. An outer surface of the inner tube 407 may touch or intimately contact an inner surface of the outer tube 409 about a full periphery of the inner tube 407 when the inner tube 407 is positioned inside the outer tube 409. Stated otherwise, there may be close fit between the inner and outer tubes 407, 409. Positioning the tube 407 within the tube 409 is not a step employed in many methods, but is discussed here to demonstrate a relationship that may exist between the tubes 407, 409 that are provided in the stages depicted in FIGS. 22A and 22B. As discussed hereafter, the inner tube 407 can be used to form inner link segments, and the outer tube 409 can be used to form outer link segments.

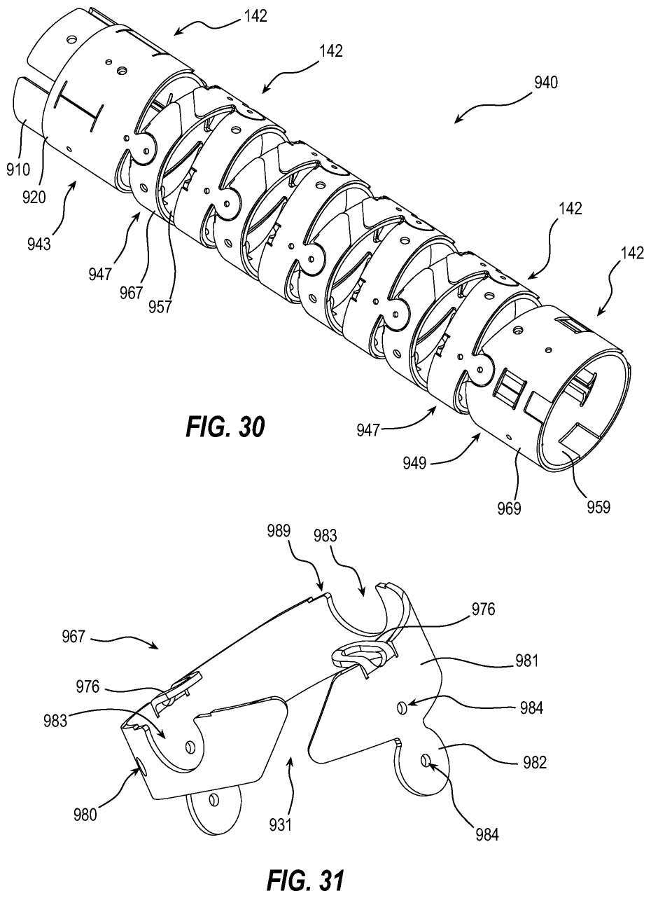

In some instances, the inner and outer tubes 407, 409 can be stock tubes that are commercially available in predetermined sizes within relatively strict tolerances. Accordingly, it may be possible to obtain inner and outer tubes 407, 409 that are capable of a close fit without any alteration of links cut therefrom. In other instances, as discussed further below with respect to FIGS. 30-41, it may be possible to use inner and outer tubes 407, 409 having outer and inner surfaces, respectively, that are not as closely matched. Indeed, in some instances, the inner and outer tubes 407, 409 may be of the same configuration, or stated otherwise, the inner tube 407 and the outer tube 409 may be of the same size. For the remainder of the discussion of the present illustrative method, however, the inner and outer tubes 407, 409 are closely matched in their outer and inner dimensions, respectively, such that tubular elements cut from the tube 407 can slide or seat within tubular elements cut from the tube 409 without alteration of such cut tubular elements.

With reference to FIG. 22C, the tube 407 is laser cut into various inner links. In the illustrated embodiment, an inner distal link or inner pull ring 459 is cut from the distal end of the tube 407, and a plurality of inner guide links 457 and inner basic links 455 are cut from the tube 407. Although multiple varieties of inner links (455, 457, 459) are depicted as being cut from the same tube 407 in the illustrated method, in other methods, each variety of inner link may be cut from separate preformed tube 407. The various preformed tubes 407 may each be of the same configuration, as compared with the other preformed tubes 407.

Moreover, with respect to the inner guide links 457 shown in FIG. 22C, the eyelets 476 that are cut initially conform to the cylindrical shape of the tube 407 from which the inner guide links 457 are cut. Thereafter, the eyelets 476 can be bent inward in any suitable pressing, folding, or bending process.

With reference to FIG. 22D, the tube 409 is laser cut into various outer links. In the illustrated embodiment, an outer distal link or outer pull ring 469 is cut from the distal end of the tube 409, and a plurality of outer basic links 465 are cut from the tube 409. Although multiple varieties of outer links are depicted as being cut from the same tube 409 in the illustrated method, in other methods, the various varieties of links may be cut from separate tubes 409.

In FIG. 23A, an inner pull ring 459 that was cut from the tube 407 is provided. In FIG. 23B, an outer pull ring 469 that was cut from the tube 409 is then advanced over the inner pull ring 459 such that alignment openings 470, 480 that extend through the sidewalls of the inner and outer pull rings 459, 469, respectively, are aligned. In FIG. 23C, an alignment pin 498 is then advanced through all of the alignment openings 470, 480.

In FIG. 23D, a basic inner link 455 is advanced longitudinally toward and laterally into coupled engagement with the inner pull ring 459, such that pivot members of the basic inner link 455 are advanced laterally into sockets defined by the inner pull ring 459. In FIG. 23E, a basic outer link 465 is then advanced over the basic inner link 455 such that locking members thereof overlie the pivot members of the basic inner link 455 and are received into recesses defined by the outer pull ring 459. Alignment openings of the inner and outer basic links 455, 465 are aligned in this process. In FIG. 23F, another alignment pin 498 is then advanced through all of the alignment openings defined by the inner and outer basic links 455, 465.

With reference to FIG. 23G, in further stages of manufacturing the backbone 440, additional inner basic links 455, with additional outer basic links 465 and alignment pins, and also inner guide links 457, with outer basic links 465 and alignment pins, are added to the assembly in manners such as depicted in FIGS. 23A-23F. The inner and outer links are then welded together at suitable welding positions, such as along adjacent edges and/or as provided by welding openings through the outer links (such as depicted, e.g., in FIGS. 30-32D). Laser welding may be used. Thereafter pull wires 121, 122, 123, 124 may be channeled through the guides (e.g., eyelets) defined by the inner guide links 457. The pull wires 121, 122, 123, 124 may be welded at distal ends thereof to the inner and/or outer pull rings 459, 469.

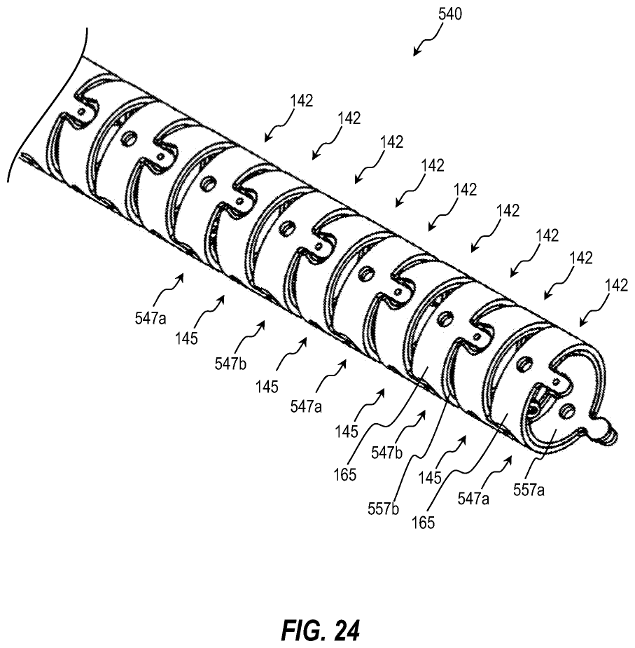

FIG. 24 is a perspective view of another embodiment of an articulable backbone 540 that is compatible with the bending section of, for example, an endoscope, such as the endoscope 100 discussed above. The backbone 540 can resemble the backbones 140, 340, 440 in many respects. In some embodiments, the backbone 540 includes numerous components similar or identical to those of one or more of the backbones 140, 340, 440. In the illustrated embodiment, the backbone 540 includes a series or chain of interconnected links 142 that can be rotated relative to one another in manners such as previously disclosed. Some of the links 142 are basic links 145, which can be substantially the same as or identical to the basic links 145 discussed above. Some of the links 142 can comprise another embodiment of guide links 547, which can differ somewhat from the guide links 147, 347 in manners such as described hereafter. For example, as further discussed below, the guide links 547 may allow for a relatively larger accessory space, as compared with certain embodiments of the guide links 147, 347, 447.

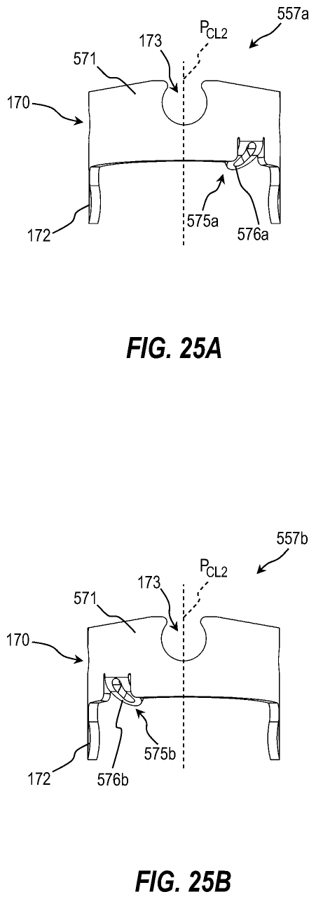

With continued reference to FIG. 24, and with reference to FIGS. 25A and 25B, in the illustrated embodiment, each guide link 547a, b includes an outer basic link 165 and one of two mirror-image varieties of an inner guide link 557a or 557b, respectively. Each inner guide link 557a, b includes a single guide 575a, b in the form of inwardly directed eyelet 576a, b. Each of the guides 575a, b is angularly spaced 45 degrees away from one of the two pivot members 172 defined by the inner guide link 557a, b, either in a first direction from a first pivot member 172 or in a second direction from a second pivot member 172 that is opposite the first direction.

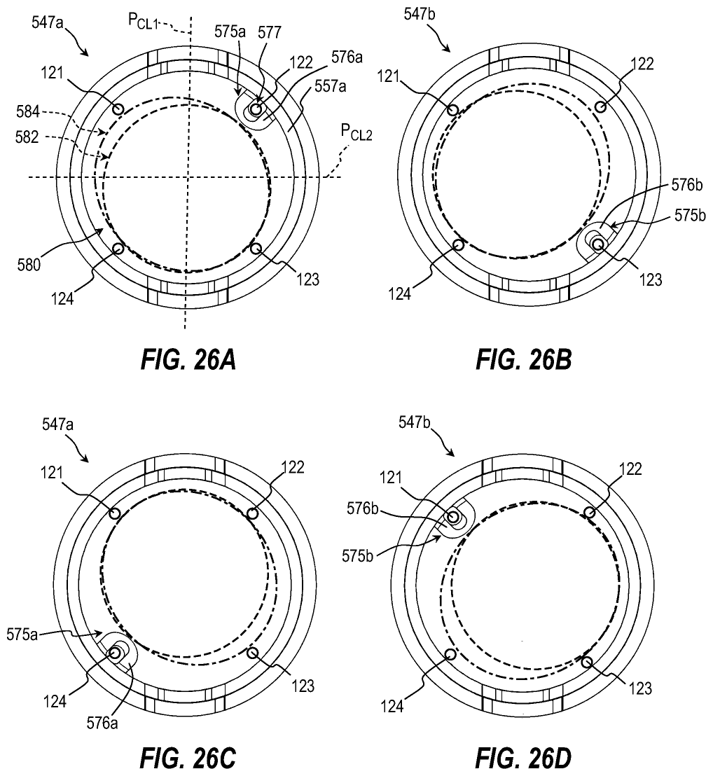

As shown in FIG. 24, for each pair of adjacent guide links 547a, b, the guide links 547a, b are separated from one another by only one intervening basic link 145. Moreover, as shown in FIGS. 26A-26D, each set of adjacent guide links 547a, b is rotated 180 degrees from an adjacent set of guide links 547a, b. Stated otherwise, pairs of adjacent guide links 547a, b are serially rotated by 180 degrees along a length of the backbone 540. In such an arrangement, the intervening basic links 145 can provide spacing within which a substantially cylindrical tubular region can gradually undulate, e.g., helically, along the longitudinal length of the backbone 540. As further discussed below, a single-guide arrangement 575a, b can facilitate use of an elliptical or oval cylinder, which may increase a cross-sectional area of usable space within the backbone 540.

Any suitable number or pattern of guide links 547a, b and basic links 145 are contemplated. Any suitable rotational pattern of adjacent guide links 547a, b is also contemplated.

With reference to FIGS. 25A and 25B, the inner guide links 557a, b each can include a body or ring 571 that includes a single ring, loop, or eyelet 576a, b that extends from a distal end of the ring 571. As with the eyelets 347, 447, the eyelets 576a, b may be laser cut and bent inward. The eyelets 576a, b can include openings 577 through which the pull wires pass. The eyelets 576a, b can function as guides 575 or retainers for the pull wires. In the illustrated embodiment, the inner guide links 557a, b each includes a single eyelets 576a, b, respectively, that are positioned at opposite sides of a central longitudinal plane P_{CL} that passes between pivot members 172 of the guide links 557a, 557b. Each eyelet 557a, b is angularly separated from a respective one of the pivot members 172 by 45 degrees.

The inner guide link 557 can include pairs of alignment openings 170, pivot members 172, and sockets 173 such as those disclosed previously. The inner guide links 557 can function substantially as described above with respect to the inner guide links 157, 357, 457.

FIG. 26A depicts a guide link 547a that is rotated such that its eyelet 576a is positioned at a first side of a first central longitudinal plane P_{CL}, and at a first side of an orthogonally oriented second longitudinal plane P_{CL2}. FIG. 26C depicts a guide link 547a of identical construction that is rotated 180 degrees relative to the guide link 547a in FIG. 21A. FIGS. 26B and 26D similarly depict two identical guide links 547b that are rotated 180 degrees relative to one another. The sequence of FIGS. 26A-26D can correspond with a longitudinal sequence in which the guide links 547a, b are provided in the backbone 540, such that a position of a single eyelet 576a, b rotates 90 degrees around a center of the backbone 540 at each level.

Each guide link 547 can define a lumen 580 such as the lumens 380, 480 previously described. In some instances, the largest possible space a cylindrical tube may occupy within the lumen 580 may be delimited by the eyelet 576a, b of that guide link 547a, b and the remaining three pull wires 121, 122, 123, 124 that are positioned outside of that eyelet 576a, b. For example, in each of FIGS. 26A-26D, an outer boundary of a cylindrical accessory space 582 (labeled in FIG. 26A) is shown. In some instances, a larger oblong, elliptical, or ovular accessory space 584 may be possible, due to the presence of a single eyelet 576a, b in each guide link 547a, b. The accessory space 584 is also labeled in FIG. 26A and shown in FIGS. 26A-26D.

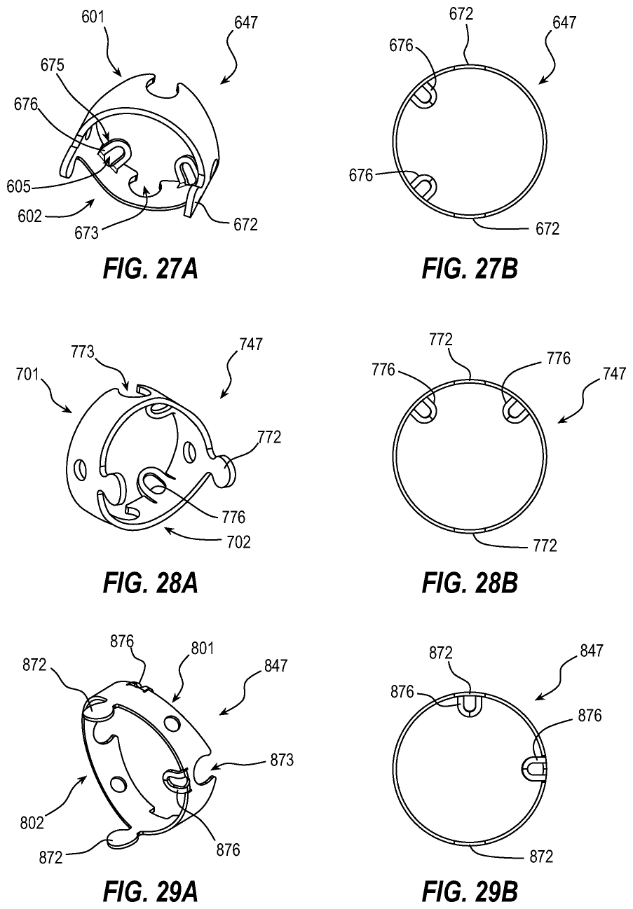

FIGS. 27A and 27B depict another embodiment of an inner guide link 647 that differs in certain respects from other inner guide links herein disclosed, such as the guide links 447, 547. The guide link 647 may nevertheless be used in certain embodiments of the backbone 540 or other backbones disclosed herein. The inner guide link 647 includes a proximal end 601 and a distal end 602, and further includes a pair of wire guides 675, a pair of sockets 673, a pair of pivot members 672, and a pair of alignment openings (shown, but not numerically identified; substantially the same as the alignment openings 170 previously discussed). The proximal and distal ends 601, 602 may be said to be at longitudinally opposite ends of the inner guide link 647.

The wire guides 675 can include any suitable wire channeling feature that provides a wire passage 605 through which a wire may pass. In the illustrated embodiment, the wire guides 675 comprises a pair of eyelets 676. In the illustrated embodiment, the eyelets 676 are positioned at the proximal end 601 of the inner guide link 647. This is in contrast to, e.g., the eyelets 476 (FIG. 20A), which are positioned at a distal end of the inner guide link 457. Moreover, the eyelets 676 are angularly spaced from each other by approximately 90 degrees, and straddle a socket 673 so as to be equally spaced therefrom. The eyelets 476, in contrast straddle the pivot member 172. Other suitable numbers, positions, and/or other configurations of the wire guides 675 are contemplated. For example, FIGS. 28A-29B depict additional illustrative suitable variations.

In FIGS. 28A and 28B, an inner guide link 747 includes, inter alia, a proximal end 701, a distal end 702, a pair of pivot members 772, and a pair of eyelets 776. The eyelets 776 are angularly spaced apart by approximately 90 degrees, and straddle or span one of the pivot members 772. The eyelets 776 are arranged similarly to the eyelets 476 of the inner guide link 457, except that they are positioned at the proximal end 701 of the inner guide link 747, rather than the distal end 702.

FIGS. 29A and 29B depict an inner guide link 847 that includes a proximal end 801 and a distal end 802. The guide link 847 includes a pair of eyelets 876. One eyelet 876 is longitudinally aligned with (e.g., has no angular spacing from) a pivot member 872, whereas the other eyelet 876 is longitudinally aligned with (e.g., has no angular spacing from) a socket 873. The eyelet 876 that is longitudinally aligned with the pivot member 872 is positioned at the proximal end 801, whereas the pivot member 872 is positioned at the distal end 802. The eyelet 876 that is longitudinally aligned with the socket 873 is positioned at the distal end 802, whereas the socket 873 is positioned at the proximal end 801.

Any suitable number of eyelets 876 and/or other wire guides is contemplated. Any suitable longitudinal and/or angular positioning of the eyelets 876 or other wire guides, e.g., relative to a socket and/or relative to pivot member is also contemplated.

FIG. 30 depicts another embodiment of an articulable backbone 940 compatible with the bending section of an elongated medical device, such as, e.g., the endoscope 100. The backbone 940 can include a plurality of links 142 of multiple varieties. In the illustrated embodiment, the backbone 940 includes three different varieties of links 142: a proximal connector link 943, a plurality of guide links 947, and a distal link or pull ring 949. The proximal connector link 943 includes an inner proximal connector link 910 and an outer proximal connector link 920. Each guide link 947 includes an inner guide link 957 and an outer guide link 967. The pull ring 949 includes an inner pull ring link 959 and an outer pull ring link 969.

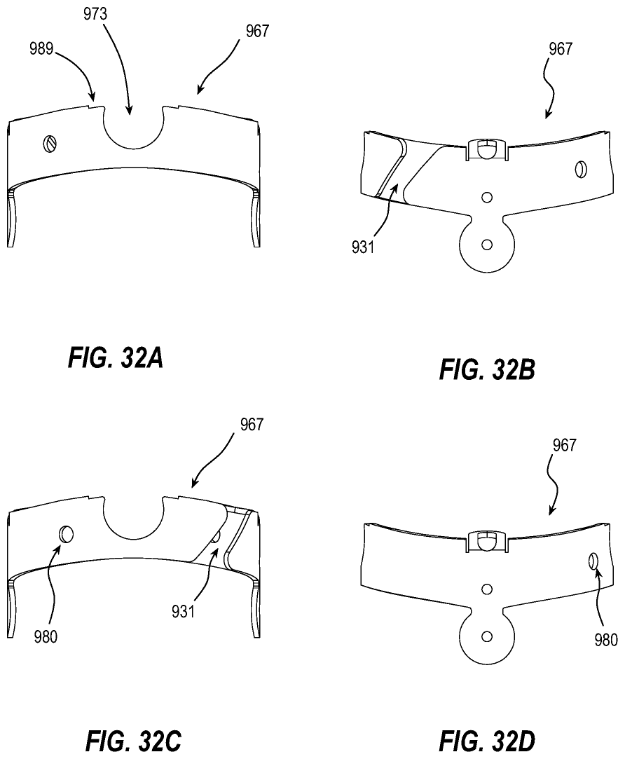

With reference to FIGS. 31-32D, the outer guide link 967 can include a body 981. Extending distally at a distal end of the body 981 are a pair of diametrically opposed locking members 982. The locking members 982 are substantially rounded and shaped much like previously described pivot members. As discussed further below, certain embodiments of the locking members 982 can be configured to overlie and fully cover pivot members of the inner guide link 957.

The outer guide link 967 can include any suitable number of welding openings 984 at which welds (e.g., laser welds) joining the outer guide link 967 to the inner guide link 957 can be made. In the illustrated embodiment, two welding openings 984 are present at the angular position of each locking member 982. A first of each set of welding openings extends through the locking member 982, such that the locking member 982 can be fixedly secured, via welding, directly to the underlying pivot member of the inner guide link 957. A further welding opening 984 of each set may be longitudinally aligned with the first welding opening 984, but spaced more proximally ate or near a medial latitude of the body 981.

In the illustrated embodiment, a pair of diametrically opposed eyelets 976 extend inwardly at an upper end of the body 981. Thus, rather than extending inwardly from an inner link, the eyelets 976 of the present embodiment extend inwardly from the external link. In the illustrated embodiment, each eyelet 976 is longitudinally aligned with one of the locking members 982.

At an orthogonal orientation relative to the locking members 982 are a pair of recesses 983 that are configured to receive locking members 982 of separate outer guide link 967. Bordering each recess 983 at a proximal end thereof are a pair of welding recesses 989. Welds can be formed at these positions to further fixedly secure the outer guide link 967 to the inner guide link 957.

The outer guide link 967 can further include alignment openings 980 such as alignment openings previously described. In some embodiments, it may be desirable to have more than two such openings. For example, it may take additional effort to ensure proper alignment of the outer guide link 967 relative to the inner guide link 957 prior to welding, such that additional weld openings (totaling three, four, five, etc.) may be used. In some instances, if four weld openings are present, each oppositely positioned pair of openings may be at a different latitude, relative to a longitudinal height of the outer guide link 967, to ensure that two separate pins may be used, each passing through a separate pair of the openings at a different height so as not to interfere with the other pin at a position interior to the assembled inner and outer guide links 957, 967.

As previously noted, in some embodiments, the outer guide link 967 can comprise an open tubular element, in contrast to a closed tubular element such as described in certain prior embodiments. In this context, an “open” tubular element is not referring to the large interior opening defined by the element, whether of an open or closed variety. Rather, “open” refers to an opening or slit that extends at least longitudinally through a sidewall of the outer guide link 967. Stated otherwise, a periphery or loop defined by the element is not continuous, but includes a gap or opening. Thus, in a closed tubular element, the element defines a tubular shape that may have a closed peripheral outline, or one without openings or gaps. The illustrated outer guide link 967 includes an opening, gap, slot, channel, separation, split, groove, spacing, or slit 931. The outer guide link 967 may be said to be an open tubular element.

As previously discussed, creating an open tubular element from a preformed tube can allow manufacture of a multi-layer link where closely fitting tubes may not be commercially available and/or commercially feasible. In some instances, preformed tubes of significantly different sizes, such that larger tubes have an inner surface that does not closely fit with an outer surface of the smaller preformed tubes, may nevertheless be used in creating outer and inner links. More generally, differently sized tubes, whether or not either snugly fits with the other, may be used to create the inner and outer links. In some instances, a same sized and/or configured preformed tube may be used for cutting out both inner and outer link segments.

Some methods of manufacture may begin with a flat sheet of material (e.g., metal), and one or more of the inner and outer links can subsequently be cut and then bent into a tubular shape. For example, in some embodiments, the inner links may be cut from a preformed tube, whereas the outer links may be cut from a sheet of material and subsequently bent or rounded to extend about a periphery of an inner link.

In the illustrated embodiment, the slit 931 extends at an angle of approximately 45 degrees relative to a longitudinal axis of the outer guide link 967. Other angular arrangements are contemplated. In some instances, it can be desirable for at least some component of the slit 931 to extend angularly around a periphery of the inner guide link 957. Such may increase an overall length of the weld and/or increase a strength of the weld, providing additional hoop strength. In other embodiments, the slit may extend purely longitudinally.

The slit 931 may be the result of a single cut or multiple cuts. For example, in some embodiments, a preformed tube is cut with, e.g., locking member 982 and recess 983 features and a single cut, and the cut region is separated to form a slit 931 with a greater width.

In some embodiments, welding may be applied along one or both edges of the slit 931 to join the outer guide link 967 to the inner guide link 957. In some instances, it may be more efficient to provide linear welds in a longitudinal direction, as minimal rotation of the welding piece and/or off-axis movement of a laser welder may be needed.

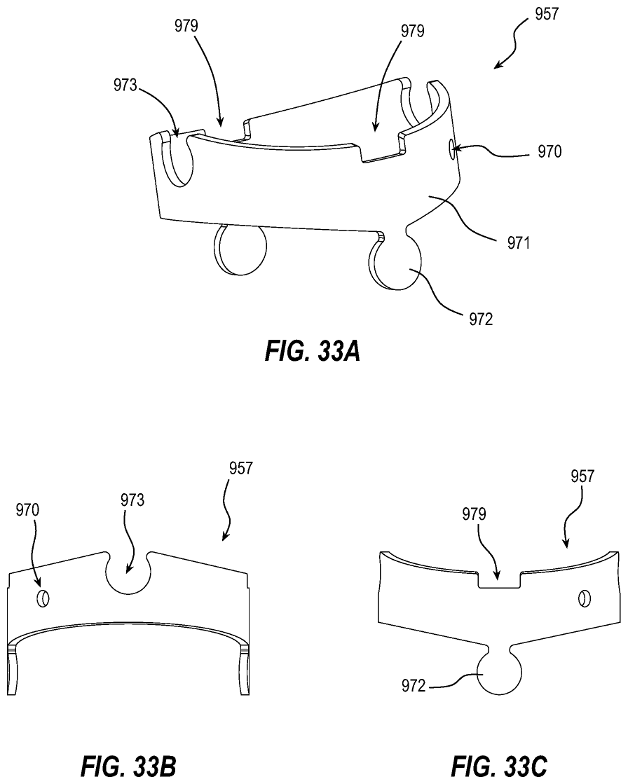

FIGS. 33A-33C depict an embodiment of the inner guide link 957. The inner guide link 957 can include features such as previously described, such as alignment openings 970, pivot members 972, and sockets 973. In the illustrated embodiment, the inner guide link 957 further includes a pair of eyelet recesses or notches 979. The notches 979 are cutout insets from a proximal face of a body 971 of the inner guide link 957. The notches 979 can provide a passageway for the bent eyelets 976 of the outer guide link 967 to pass to an interior of the assembled backbone 940.

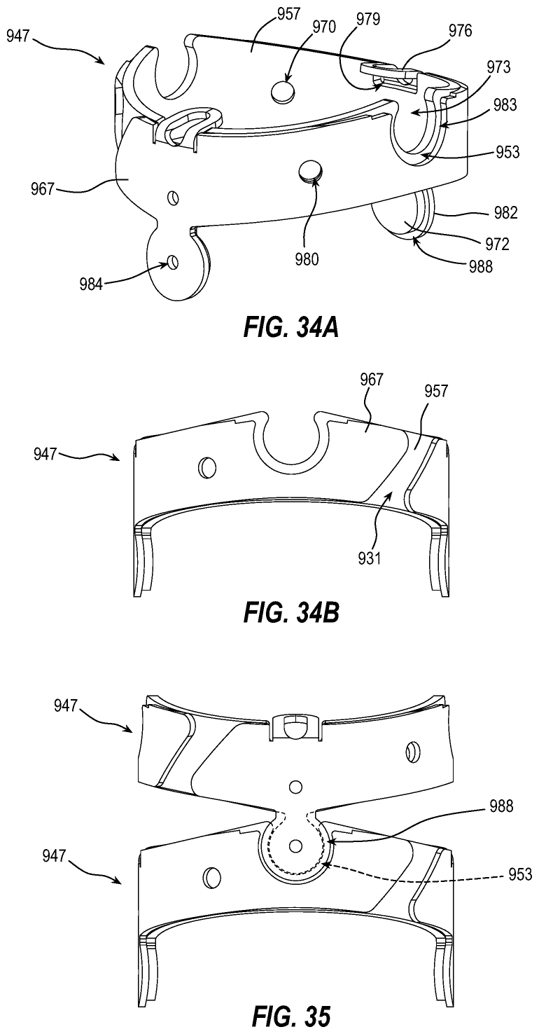

Such an arrangement of the assembled guide link 947 is depicted in FIGS. 34A and 34B. As shown, the eyelets 976 extend through the notches 979. As with previous embodiments, the alignment openings 980, 970 are aligned to assist in fixed securement of the link segments to each other.

With reference to FIGS. 34A-35, similar to previously described embodiments, each guide link 947 can include a pair of locking surfaces 953 and a corresponding pair of locking sections 188. Each locking surface 953 can be that portion of the outer surface of the inner guide link positioned between an edge of a socket 973 and an edge of a recess 983. An inner peripheral portion of each locking member 982 can define the locking sections 988. As previously discussed, the locking sections 988 and the locking surfaces 953 can interact to maintain adjacent links 947 in a coupled configuration by inhibiting or preventing lateral movement therebetween.

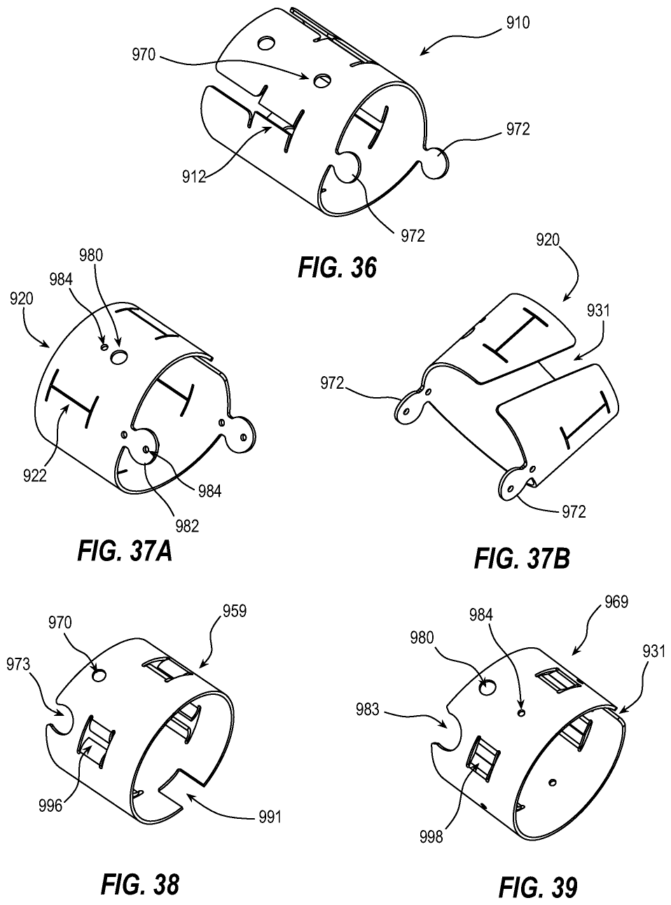

With reference to FIG. 36, the inner proximal connector link 910 can be configured to couple with other elements of an elongated medical shaft. For example, in some embodiments, the inner proximal connector link 910 can be welded to compression coils through which the pull wires extend along a proximal portion of the shaft. The inner proximal connector link 910 can include welding windows 912 for making such connections. As with other two-layer link systems described herein, the inner link 910 can include one or more alignment openings 970. The inner link 910 may include a pair of pivot members 972 for coupling with an adjacent guide link 947 in manners such as previously described.

With reference to FIGS. 37A and 37B, the outer proximal connector link 920 can include welding openings 984 and alignment openings 980. It may further include a pair of locking members 982 that can function in manners such as previously described. The outer proximal connector link 920 may include a slit 931, which may function in manners such as previously described.

With reference to FIG. 38, the inner distal link or inner pull ring 959 can be configured to couple with other elements of an elongated medical shaft. For example, in some embodiments, the inner pull ring 959 can be joined to a distal tip of the medical instrument, which may include camera, lighting, and/or other componentry. In the illustrated embodiment, the inner pull ring 959 includes a clocking recess 991 to assist with proper alignment of such a distal tip. The inner pull ring 959 may further be welded to distal ends of the pull wires. The inner pull ring 959 can include welding windows 996 for making such connections. As with other two-layer link systems described herein, the inner pull ring 959 can include one or more alignment openings 970. The inner pull ring 959 may include a pair of sockets 973 for coupling with an adjacent guide link 947 in manners such as previously described.

With reference to FIG. 39, the outer distal link or outer pull ring 969 can include welding openings 998 and alignment openings 980. It may further include a pair of locking members recesses 983 that can interface with locking members of an adjacent guide link 947 and function in manners such as previously described. The outer pull ring 969 may include a slit 931, which may function in manners such as previously described.

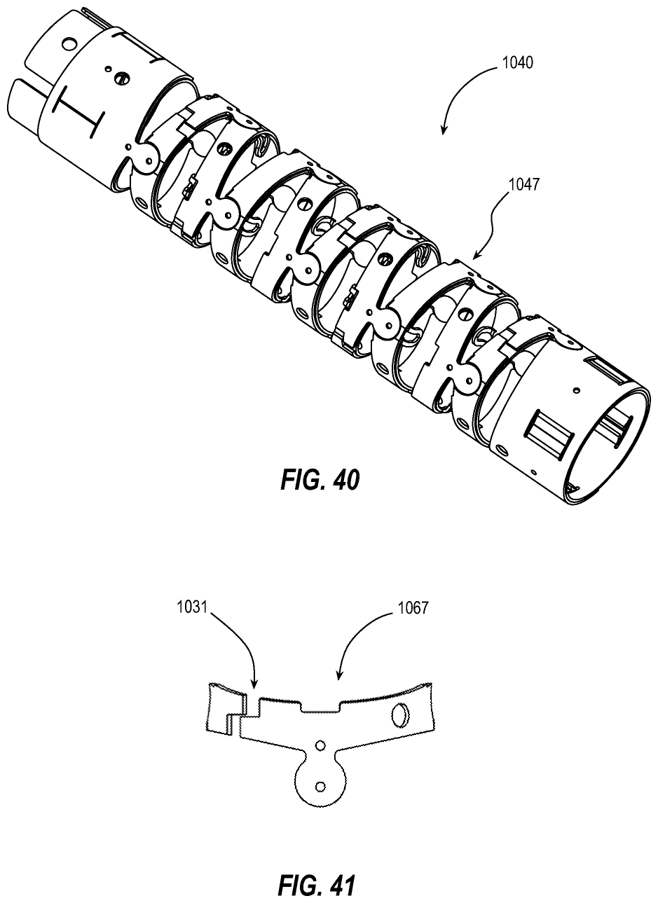

FIG. 40 depicts another embodiment of an articulable backbone 1040 compatible with the bending section of an elongated medical device, such as, e.g., the endoscope 100. The backbone 1040 can include a plurality of links of multiple varieties, similar to the backbone 940 just described.

As shown in FIGS. 40 and 41, in the illustrated embodiment, the backbone 940 includes a different variety of guide link 1047, in that an outer guide link 1067 may differ from the outer guide link 967. In particular, the outer guide link 1067 includes a slit 1931 of a different configuration than the slit 931. The slit 1931 includes two longitudinal openings or chasms that are joined by a lateral opening or chasm. The slit 1931 may be formed by a cut that extends along a first longitudinal length, proceeds around a portion of the circumference at a right angle to the first longitudinal length, and then proceeds along a second longitudinal length. The cut may be separated to form the widened slit 1931. As previously discussed, such an arrangement may permit welding along one or more peripheral lines, which may increase an overall hoop strength of the guide link 1047.

Other slit arrangements are contemplated.

Various illustrative embodiments and examples are described in the following Clauses, each being a separate embodiment. Each recited embodiment is supported by the foregoing written description and accompanying drawings and/or provides independent support for the recited features in view of the same.

Clause 1. A backbone for use in an elongated medical instrument, the backbone comprising:
a first link comprising:
   a first inner link segment that comprises a pivot member; and
   a first outer link segment fixedly secured to the first inner link segment, the first outer link segment comprising a locking member; and
a second link comprising:
   a second inner link segment that comprises a socket; and a second outer link segment fixedly secured to the second inner link segment, the second outer link segment comprising a recess;
wherein the pivot member of the first inner link segment is positioned within the socket of the second inner link segment such that the pivot member is rotatable within the socket to permit the first link to rotate relative to the second link, and
wherein the locking member of the first outer link segment is positioned within the recess of the second outer link segment to inhibit lateral movement of the first and second links relative to each other and thereby maintain the pivot member of the first inner link segment within the socket of the second inner link segment.

Clause 2. The backbone of Clause 1, wherein each of the first and second inner link segments is fixedly secured to the first and second outer link segments, respectively, via one or more welds.

Clause 3. The backbone of Clause 1, wherein the first inner link segment comprises a first closed tubular element and the second inner link segment comprises a second closed tubular element.

Clause 4. The backbone of Clause 3, wherein the first outer link segment comprises a third closed tubular element that fully encircles the first closed tubular element of the first inner link segment.

Clause 5. The backbone of Clause 4, wherein the second outer link segment comprises a fourth closed tubular element that fully encircles the second closed tubular element of the second inner link segment.

Clause 6. The backbone of Clause 3, wherein the first outer link segment comprises a first open tubular element that comprises a first slit, and wherein the first open tubular element encompasses the first closed tubular element of the first inner link segment without extending about an entirety of a periphery of the first closed tubular element.

Clause 7. The backbone of Clause 6, wherein the second outer link segment comprises a second open tubular element that comprises a second slit, and wherein the second open tubular element encompasses the second closed tubular element of the second inner link segment without extending about an entirety of a periphery of the second closed tubular element.

Clause 8. The backbone of Clause 1, wherein the first inner link segment further comprises an additional socket.

Clause 9. The backbone of Clause 8, wherein the pivot member and the additional socket of the first inner link segment are positioned at opposite longitudinal ends of the first inner link segment.

Clause 10. The backbone of Clause 9, wherein the pivot member and the additional socket of the first inner link segment are angularly offset from one another.

Clause 11. The backbone of Clause 10, wherein the pivot member and the additional socket of the first inner link segment are angularly offset from one another by 90 degrees.

Clause 12. The backbone of Clause 1, wherein the first outer link segment further comprises an additional recess.

Clause 13. The backbone of Clause 12, wherein the locking member and the additional recess of the first outer link segment are positioned at opposite longitudinal ends of the first outer link segment.

Clause 14. The backbone of Clause 13, wherein the locking member and the additional recess of the first outer link segment are angularly offset from one another.

Clause 15. The backbone of Clause 14, wherein the locking member and the additional recess of the first outer link segment are angularly offset from one another by 90 degrees.

Clause 16. The backbone of Clause 1, wherein the first link further comprises a wire passage configured to receive a pull wire therethrough.

Clause 17. The backbone of Clause 16, wherein the wire passage is at least partially defined by a channel that extends between the first inner link segment and the first outer link segment.

Clause 18. The backbone of Clause 16, wherein the first inner link segment comprises an eyelet that defines the wire passage, the eyelet extending inwardly into an interior of the backbone.

Clause 19. The backbone of Clause 16, wherein the first outer link segment comprises an eyelet that defines the wire passage, the eyelet extending inwardly into an interior of the backbone.

Clause 20. The backbone of Clause 19, wherein the first inner link segment comprises a notch within which at least a portion of the eyelet is positioned.

Clause 21. The backbone of Clause 1, wherein the first inner link segment defines a first alignment opening and the first outer link segment defines a second alignment opening that cooperates with the first alignment opening to define an alignment passageway through a sidewall of the first link.

Clause 22. The backbone of Clause 1, wherein the first outer link segment defines a welding opening at which the first outer link segment is welded to the first inner link segment.

Clause 23. A medical instrument that comprises:
an elongated shaft; and
the backbone of Clause 1 incorporated into the elongated shaft.

Clause 24. The medical instrument of Clause 23, wherein the medical instrument is an endoscope.

Clause 25. A link for use in a bending section of an elongated medical instrument, the link comprising:
an inner link segment that comprises a pivot member and a socket; and
an outer link segment fixedly secured to the inner link segment, the outer link segment comprising:
   a locking member that overlies at least a portion of the pivot member of the inner link segment, wherein a locking section of the locking member extends beyond a peripheral edge of the pivot member; and
   a recess that extends around at least a portion of the socket, the recess comprising a peripheral edge that is spaced from a peripheral edge of the socket, thereby leaving a locking surface of the inner link segment exposed.

Clause 26. The link of Clause 25, wherein the locking surface is substantially arc-shaped.

Clause 27. The link of Clause 25, wherein the locking member is narrower than a maximum width of the pivot member.

Clause 28. The link of Clause 25, wherein the locking member overlies an entirety of the pivot member.

Clause 29. The link of Clause 25, wherein the locking member comprises a rounded profile that is substantially complementary in shape to a profile of the recess.

Clause 30. The link of Clause 25, wherein the pivot member and the socket are positioned at opposite ends of the inner link segment.

Clause 31. The link of Clause 30, wherein the pivot member and the socket are angularly offset from one another by 90 degrees.

Clause 32. The link of Clause 25, wherein the locking member and the recess are positioned at opposite ends of the outer link segment.

Clause 33. The link of Clause 32, wherein the locking member and the recess are angularly offset from one another by 90 degrees.

Clause 34. An apparatus comprising the link of Clause 25 and an additional link that comprises an additional locking member that is positioned within the recess of the outer link segment, the additional locking member comprising an additional locking section that overlies at least a portion of the locking surface of the inner link segment.

Clause 35. An apparatus comprising the link of Clause 34, wherein interference between the additional locking section and the locking surface of the inner link segment inhibits lateral movement of the links relative to each other.

Clause 36. An apparatus comprising the link of Clause 34, wherein the additional link further comprises an additional pivot member that is positioned within the socket of the inner link segment, the additional pivot member being configured to rotate within the socket.

Clause 37. The apparatus of Clause 36, wherein the additional locking section overlies at least a portion of the additional pivot member.

Clause 38. An apparatus comprising the link of Clause 25 and an additional link that comprises an additional pivot member that is positioned within the socket of the inner link segment, the additional pivot member being configured to rotate within the socket.

Clause 39. A link for use in a bending section of an elongated medical instrument, the link comprising:
an inner link segment that comprises a pair of pivot members and a pair of sockets; and
an outer link segment fixedly secured to the inner link segment, the outer link segment comprising:
   a pair of locking members, each of which overlies at least a portion of a respective one of the pivot members of the inner link segment; and
   a pair of recesses, each of which extends around at least a portion of a respective one of the sockets.

Clause 40. A method comprising:
cutting, from a first preformed tube, a first inner link segment that comprises a first pivot member and a first socket;
cutting, from a second preformed tube, a first outer link segment that comprises a first locking member and a first recess;
positioning the first inner link segment within the first outer link segment such that the first recess at least partially encompasses the first socket and the first locking member overlies at least a portion of the first pivot member; and
fixedly securing the first outer link segment to the first inner link segment.

Clause 41. The method of Clause 40, wherein the first preformed tube comprises an outer surface and the second preformed tube comprises an inner surface, the outer surface being configured to fit snugly within the inner surface.

Clause 42. The method of Clause 40, wherein each of the first and second preformed tubes is substantially cylindrical.

Clause 43. The method of Clause 40, wherein an outer diameter of the first preformed tube is slightly smaller than an inner diameter of the second preformed tube such that an outer surface of the first preformed is configured to make intimate contact with an inner surface of the second preformed tube when the first preformed tube is positioned inside of the second preformed tube.

Clause 44. The method of Clause 40, wherein cross-sectional dimensions of the first and preformed tube differ from cross-sectional dimensions of the second preformed tube.

Clause 45. The method of Clause 40, wherein cross-sectional dimensions of the first and second preformed tubes are substantially identical.

Clause 46. The method of Clause 40, wherein said cutting from each of the first and second preformed tubes comprises laser cutting.

Clause 47. The method of Clause 40, wherein said cutting of the first outer link segment from the second preformed tube comprises making a cut that extends from a first end to a second end of the first outer link.

Clause 48. The method of Clause 47, wherein said positioning the first inner link segment within the first outer link segment comprises widening the cut that extends from the first end to the second end of the first outer link to form a slit.

Clause 49. The method of Clause 48, further comprising encompassing the first outer link segment about the first inner link segment without fully encircling the first inner link segment.

Clause 50. The method of Clause 40, wherein the first preformed tube comprises a first metallic hypotube and the second preformed tube comprises a second preformed hypotube.

Clause 51. The method of Clause 40, further comprising:
forming an alignment opening in the first inner link segment;
forming an alignment opening in the first outer link segment;
aligning, during said positioning of the first inner link segment within the first outer link segment, the alignment openings; and
advancing an alignment pin through the alignment openings prior to said fixedly securing the first outer link segment to the first inner link segment.

Clause 52. The method of Clause 51, further comprising:
forming an additional alignment opening in the first inner link segment;
forming an additional alignment opening in the first outer link segment;
aligning, during said positioning of the first inner link segment within the first outer link segment, the additional alignment openings; and
advancing the alignment pin through the additional alignment openings, such that the alignment pin extends through all of the alignment openings, prior to said fixedly securing the first outer link segment to the first inner link segment.

Clause 53. The method of Clause 40, further comprising:
forming a second inner link segment that comprises a second pivot member and a second socket; and
positioning the second pivot member of the second inner link segment within the first socket of the first inner link segment.

Clause 54. The method of Clause 53, wherein said positioning of the second pivot member of the second inner link segment within the first socket of the first inner link segment takes place prior to said fixedly securing the first outer link segment to the first inner link segment.

Clause 55. The method of Clause 53, further comprising:
forming a second outer link segment that comprises a second locking member and a second recess; and
positioning the second locking member of the second outer link segment within the first recess of the first outer link segment.

Clause 56. The method of Clause 40, further comprising incorporating the first inner link segment and the first outer link segment into an elongated shaft of a medical device.

Clause 57. A method comprising:
cutting, from a first preformed tube, a first inner link segment that comprises a closed tubular element;
cutting, from a second preformed tube, a first outer link segment;
forming a slit in the first outer link segment that extends from a first end to a second end of the first outer link segment, such that the first outer link segment comprises an open tubular element;
encompassing the first outer link segment about the first inner link segment without fully encircling the first inner link segment; and
fixedly securing the first outer link segment to the first inner link segment.

Clause 58. A method comprising:
cutting, from a first preformed tube, a first inner link segment that comprises a closed tubular element;
forming a first outer link segment that includes a slit extending from a first end to a second end of the first outer link segment;
encompassing the first outer link segment about the first inner link segment without fully encircling the first inner link segment; and fixedly securing the first outer link segment to the first inner link segment.

Clause 59. A backbone for use in an endoscope, the backbone comprising:
a plurality of links of which adjacent pairs are interconnected with each other, each of the plurality of links comprising:
   an inner link segment; and
   an outer link segment fixedly secured to the inner link segment;
one of the inner link segment and the outer link segment comprising a pivot member and a socket; and
the other of the inner link segment and the outer link segment comprising a locking member and a recess,
wherein the pivot member of a first link of the plurality of links is received into the socket of a second link of the plurality of links; and
wherein the locking member of the first link is received into the recess of the second link.

Clause 60. The backbone of Clause 58, wherein inner link segment and the outer link segment are fixedly secured via welding.

Clause 61. The backbone of Clause 58, wherein the inner link segment comprises the pivot member and the socket and the outer link segment comprises the locking member and the recess.

Clause 62. An endoscope comprising a bending section into which the backbone of Clause 58 is incorporated.

The claims following this written disclosure are hereby expressly incorporated into the present written disclosure, with each claim standing on its own as a separate embodiment. This disclosure includes all permutations of the independent claims with their dependent claims. Moreover, additional embodiments capable of derivation from the independent and dependent claims that follow are also expressly incorporated into the present written description. These additional embodiments are determined by replacing the dependency of a given dependent claim with the phrase “any of claims [x] through the immediately preceding claim,” where the bracketed term “[x]” is replaced with the number of the most recently recited independent claim. For example, for the first claim set that begins with independent claim 1, claim 3 can depend from either of claims 1 and 2, with these separate dependencies yielding two distinct embodiments; claim 4 can depend from any one of claims 1, 2, or 3, with these separate dependencies yielding three distinct embodiments; claim 5 can depend from any one of claims 1, 2, 3, or 4, with these separate dependencies yielding four distinct embodiments; and so on.

Recitation in the claims of the term “first” with respect to a feature or element does not necessarily imply the existence of a second or additional such feature or element. Embodiments of the invention in which an exclusive property or privilege is claimed are defined as follows.

1. A backbone for use in an elongated medical instrument, the backbone comprising:
a first link comprising:
a first inner link segment that comprises a pivot member; and
a first outer link segment fixedly secured to the first inner link segment, the first outer link segment comprising a locking member; and
a second link comprising:
a second inner link segment that comprises a socket; and
a second outer link segment fixedly secured to the second inner link segment, the second outer link segment comprising a recess;
wherein the pivot member of the first inner link segment is positioned within the socket of the second inner link segment such that the pivot member is rotatable within the socket to permit the first link to rotate relative to the second link, and
wherein the locking member of the first outer link segment is positioned within the recess of the second outer link segment to inhibit lateral movement of the first and second links relative to each other and thereby maintain the pivot member of the first inner link segment within the socket of the second inner link segment.

2. The backbone of claim 1, wherein each of the first and second inner link segments is fixedly secured to the first and second outer link segments, respectively, via one or more welds.

3. The backbone of claim 1, wherein the first inner link segment comprises a first closed tubular element and the second inner link segment comprises a second closed tubular element.

4. The backbone of claim 3, wherein the first outer link segment comprises a third closed tubular element that fully encircles the first closed tubular element of the first inner link segment.

5. The backbone of claim 4, wherein the second outer link segment comprises a fourth closed tubular element that fully encircles the second closed tubular element of the second inner link segment.

6. The backbone of claim 3, wherein the first outer link segment comprises a first open tubular element that comprises a first slit, and wherein the first open tubular element encompasses the first closed tubular element of the first inner link segment without extending about an entirety of a periphery of the first closed tubular element.

7. The backbone of claim 6, wherein the second outer link segment comprises a second open tubular element that comprises a second slit, and wherein the second open tubular element encompasses the second closed tubular element of the second inner link segment without extending about an entirety of a periphery of the second closed tubular element.

8. The backbone of claim 1, wherein the first inner link segment further comprises an additional socket.

9. The backbone of claim 8, wherein the pivot member and the additional socket of the first inner link segment are positioned at opposite longitudinal ends of the first inner link segment.

10. The backbone of claim 9, wherein the pivot member and the additional socket of the first inner link segment are angularly offset from one another.

11. The backbone of claim 10, wherein the pivot member and the additional socket of the first inner link segment are angularly offset from one another by 90 degrees.

12. The backbone of claim 1, wherein the first outer link segment further comprises an additional recess.

13. The backbone of claim 12, wherein the locking member and the additional recess of the first outer link segment are positioned at opposite longitudinal ends of the first outer link segment.

14. The backbone of claim 13, wherein the locking member and the additional recess of the first outer link segment are angularly offset from one another.

15. The backbone of claim 14, wherein the locking member and the additional recess of the first outer link segment are angularly offset from one another by 90 degrees.

16. The backbone of claim 1, wherein the first link further comprises a wire passage configured to receive a pull wire therethrough.

17. The backbone of claim 16, wherein the wire passage is at least partially defined by a channel that extends between the first inner link segment and the first outer link segment.

18. The backbone of claim 16, wherein the first inner link segment comprises an eyelet that defines the wire passage, the eyelet extending inwardly into an interior of the backbone.

19. The backbone of claim 16, wherein the first outer link segment comprises an eyelet that defines the wire passage, the eyelet extending inwardly into an interior of the backbone.

20. The backbone of claim 19, wherein the first inner link segment comprises a notch within which at least a portion of the eyelet is positioned.

21. The backbone of claim 1, wherein the first inner link segment defines a first alignment opening and the first outer link segment defines a second alignment opening that cooperates with the first alignment opening to define an alignment passageway through a sidewall of the first link.

22. The backbone of claim 1, wherein the first outer link segment defines a welding opening at which the first outer link segment is welded to the first inner link segment.

23. A medical instrument that comprises:
an elongated shaft; and
the backbone of claim 1 incorporated into the elongated shaft.

24. The medical instrument of claim 23, wherein the medical instrument is an endoscope.
